SEQUENCE LISTING (1 sequence)

SEQ ID NO: 1 - protein, 631 aa, Hepatitis C virus
APITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVT

(19) United States
(12) Patent Application Publication
(10) Pub. No.: US 2009/0099186 A1
(43) Pub. Date: Apr. 16, 2009

(54) INHIBITORS OF VIRAL REPLICATION

(76) Inventors: Leonid Beigelman, San Mateo, CA (US); Steven W. Andrews, Longmont, CO (US); Kevin R. Condroski, Lafayette, CO (US); Indrani Gunawaradana, Longmont, CO (US); Julia Haas, Boulder, CO (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/089,903

(22) PCT Filed: Oct. 10, 2006
(86) PCT No.: PCT/US06/39044

§ 371 Date: Sep. 22, 2008

Related U.S. Application Data
(60) Provisional application No. 60/725,584, filed on Oct. 11, 2005.

(51) Int. Cl. A61K 31/535; C07C 233/00; A61K 31/165; C07D 209/44; A61K 31/40; C07D 211/60; C12N 5/02; C07D 265/30; A61K 31/445; A61K 31/454; C07D 405/06; A61K 31/496

(52) U.S. Cl. 514/237.5; 564/182; 514/617; 548/470; 514/416; 546/245; 514/315; 514/326; 544/379; 514/254.1; 544/106; 435/375

(57) ABSTRACT

The embodiments provide compounds of the general Formulas I-IV, as well as compositions, including pharmaceutical compositions, comprising a subject compound. The embodiments further provide treatment methods, including methods of treating a hepatitis C virus infection and methods of treating liver fibrosis, the methods generally involving administering to an individual in need thereof an effective amount of a subject compound or composition.

### RELATED APPLICATION

This application claims the benefit of U.S. Provisional Application No. 60/725,584, filed Oct. 11, 2005, which is incorporated herein by reference in its entirety.

### BACKGROUND OF THE INVENTION

1. Field of the Invention

The present invention relates to compounds, processes for their syntheses, compositions and methods for the treatment of hepatitis C virus (HCV) infection.

2. Description of the Related Art

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with combination therapy using pegylated IFN-α plus ribavirin, 40% to 50% of patients fail therapy, i.e., are nonresponders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

HCV is an enveloped positive strand RNA virus in the Flaviviridae family. The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins of the virus. In the case of HCV, the generation of mature nonstructural proteins (NS2, NS3, NS4, NS4A, NS4B, NS5A, and NS5B) is effected by two viral proteases. The first viral protease cleaves at the NS2-NS3 junction of the polyprotein. The second viral protease is serine protease contained within the N-terminal region of NS3 (herein referred to as “NS3 protease”). NS3 protease mediates all of the subsequent cleavage events at sites downstream relative to the position of NS3 in the polyprotein (i.e., sites located between the C-terminus of NS3 and the C-terminus of the polyprotein). NS3 protease exhibits activity both in cis, at the NS3-NS4 cleavage site, and in trans, for the remaining NS4A-NS4B, NS4B-NS5A, and NS5A-NS5B sites. The NS4A protein is believed to serve multiple functions, acting as a cofactor for the NS3 protease and possibly assisting in the membrane localization of NS3 and other viral replicase components. Apparently, the formation of the complex between NS3 and NS4A is necessary for NS3-mediated processing events and enhances proteolytic efficiency at all sites recognized by NS3. The NS3 protease also exhibits nucleoside triphosphatase and RNA helicase activities. NS5B is an RNA-dependent RNA polymerase involved in the replication of HCV RNA.

### LITERATURE

METAVIR (1994) Hepatology 20:15-20; Brunt (2000) Hepatol. 31:241-246; Alpini (1997) J. Hepatol. 27:371-380; Baroni et al. (1996) Hepatol. 23:1189-1199; Czaja et al. (1989) Hepatol. 10:795-800; Grossman et al. (1998) J. Gastroenterol. Hepatol. 13:1058-1060; Rockey and Chung (1994) J. Invest. Med. 42:660-670; Sakaida et al. (1998) J. Hepatol. 28:471-479; Shi et al. (1997) Proc. Natl. Acad. Sci. USA 94:10663-10668; Baroni et al. (1999) Liver 19:212-219; Lortat-Jacob et al. (1997) J. Hepatol. 26:894-903; Llorent et al. (1996) J. Hepatol. 24:555-563; U.S. Pat. No. 5,082,659; European Patent Application EP 294,160; U.S. Pat. No. 4,806,347; Balish et al. (1992) J. Infect. Diseases 166:1401-1403; Katayama et al. (2001) J. Viral Hepatitis 8:180-185; U.S. Pat. No. 5,082,659; U.S. Pat. No. 5,190,751; U.S. Pat. No. 4,806,347; Wandl et al. (1992) Br. J. Haematol. 81:516-519; European Patent Application No. 294,160; Canadian Patent No. 1,321,348; European Patent Application No. 276,120; Wandl et al. (1992) Sem. Oncol. 19:88-94; Balish et al. (1992) J. Infectious Diseases 166:1401-1403; Van Dijk et al. (1994) Int. J. Cancer 56:262-268; Sundmacher et al. (1987) Current Eye Res. 6:273-276; U.S. Pat. Nos. 6,172,046; 6,245,740; 5,824,784; 5,372,808; 5,980,884; published international patent applications WO 96/21468; WO 96/11953; WO 00/59929; WO 00/66623; WO2003/064416; WO2003/064455; WO2003/064456; WO 97/06804; WO 98/17679; WO 98/22496; WO 97/43310; WO 98/46597; WO 98/46630; WO 99/07733; WO 99/07734, WO 00/09543; WO 00/09558; WO 99/38888; WO 99/64442; WO 99/50230; WO 95/33764; Torre et al. (2001) J. Med. Virol. 64:455-459; Bekkering et al. (2001) J. Hepatol. 34:435-440; Zeuzem et al. (2001) Gastroenterol. 120:1438-1447; Zeuzem (1999) J. Hepatol. 31:61-64; Keeffe and Hollinger (1997) Hepatol. 26:101 S-107S; Wills (1990) Clin. Pharmacokinet. 19:390-399; Heathcote et al. (2000) New Engl. J. Med. 343:1673-1680; Husa and Husova (2001) Bratisl. Lek. Listy 102:248-252; Glue et al. (2000) Clin. Pharmacol. 68:556-567; Bailon et al. (2001) Bioconj. Chem. 12:195-202; and Neumann et al. (2001) Science 282:103; Zalipsky (1995) Adv. Drug Delivery Reviews S. 16, 157-182; Mann et al. (2001) Lancet 358:958-965; Zeuzem et al. (2000) New Engl. J. Med. 343:1666-1672; U.S. Pat. Nos. 5,633,388; 5,866,684; 6,018,020; 5,869,253; 6,608,027; 5,985,265; 5,908,121; 6,177,074; 5,985,263; 5,711,944; 5,382,657; and 5,908,121; Osborn et al. (2002) J. Pharmacol. Exp. Therap. 303:540-548; Sheppard et al. (2003) Nat. Immunol. 4:63-68; Chang et al. (1999) Nat. Biotechnol. 17:793-797; Adolf (1995) Multiple Sclerosis 1 Suppl. 1:S44-S47; Chu et al., Tet. Lett. (1996), 7229-7232; Ninth Conference on Antiviral Research, Urabandai, Fukyshima, Japan (1996) (Antiviral Research, (1996), 30:1, A23 (abstract 19)); Steinkuhler et al., Biochem., 37: 8899-8905; Ingallinella et al., Biochem., 37: 8906-8914; and U.S. Pat. No. 6,183,121, which is hereby incorporated by reference in its entirety.

### SUMMARY OF THE INVENTION

Preferred embodiments provide for a compound of the formula (I):

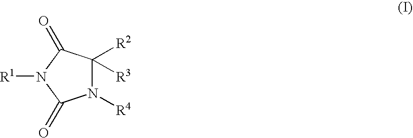

wherein:

R¹ is an optionally substituted aryl, an optionally substituted heterocyclyl comprising at least one of N, O or S, optionally substituted arylalkyl, or an optionally substituted heterocyclylalkyl comprising at least one of N, O or S in the heterocyclyl system;

R², R³ and R⁴ are each individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₆ to C₂₀ arylalkyl, optionally substituted C₃ to C₂₀ cycloalkylalkyl, optionally substituted C₅ to C₂₀ heteroarylalkyl, optionally substituted C₃ to C₂₀ heterocycylalkyl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; or

at least two of R², R³ and R⁴ join to form a ring wherein the ring is an unsubstituted or substituted 3 to 20 membered ring, wherein the members of the ring are selected from the group consisting of carbon, nitrogen, oxygen, and sulfur;

wherein formula (I) does not include the following structure:

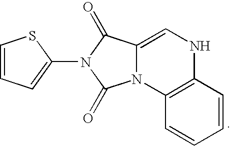

Preferred embodiments provide for a compound of the formula (II):

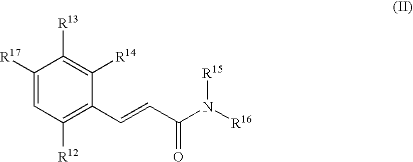

wherein:

R¹², R¹³, R¹⁴, and R¹⁷ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; wherein not all of R¹², R¹³, R¹⁴, and R¹⁷ are H;

R¹⁵ and R¹⁶ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₃ to C₂₀ heterocyclylalkyl, optionally substituted C₅ to C₂₀ heteroarylalkyl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, mono- and di-(C₁ to C₂₀)alkylamino, carbamyl, keto, carbonyl, carboxy, glycolyl, glycyl, hydrazino, guanylyl, and combinations thereof; or

R¹⁵ and R¹⁶ together form a ring wherein the ring is an unsubstituted or substituted 3 to 7 membered ring, wherein the members of the ring are selected from the group consisting of carbon, nitrogen, oxygen, or sulfur;

wherein formula (II) does not include the following structures:

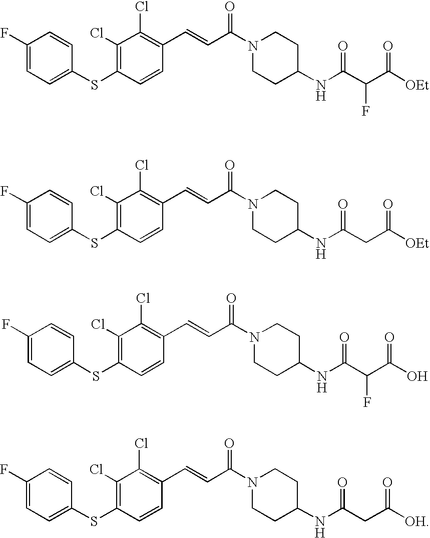

Preferred embodiments provide for a compound of the formula (IV):

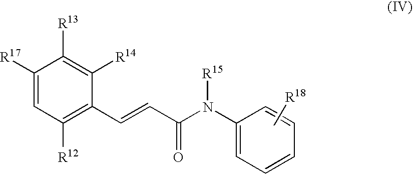

wherein:

R¹², R¹³, R¹⁴, and R¹⁷ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; wherein not all of R¹², R¹³, R¹⁴, and R¹⁷ are H;

R¹⁵ is selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₁ to C₂₀ heterocyclylalkyl, optionally substituted C₅ to C₂₀ heteroarylalkyl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, mono- and di-(C₁ to C₂₀)alkylamino, carbamyl, keto, carbonyl, carboxy, glycolyl, glycyl, hydrazino, guanylyl, and combinations thereof;

R¹⁸ is selected from the group consisting of H, optionally substituted C₁ to C₂₀ alkyl, optionally substituted C₁ to C₂₀ alkoxy, mono- and di-(C₁ to C₂₀)alkylamino, carbamyl, keto, carbonyl, carboxy, glycolyl, glycyl, hydrazino, guanylyl, and combinations thereof.

Preferred embodiments provide for a method of modulating NS3 activity comprising contacting an NS3 protein with a compound disclosed herein.

Preferred embodiments provide for a method of treating hepatitis by modulating NS3 helicase comprising contacting an NS3 helicase with the compound disclosed herein.

Preferred embodiments provide for a compound that can bind to a site of NS3 helicase and inhibit unwinding of a nucleic acid substrate, thereby modulating activity of NS3 helicase.

### DETAILED DESCRIPTION OF THE PREFERRED EMBODIMENT

### Definitions

As used herein, the term “hepatic fibrosis,” used interchangeably herein with “liver fibrosis,” refers to the growth of scar tissue in the liver that can occur in the context of a chronic hepatitis infection.

The terms “individual,” “host,” “subject,” and “patient” are used interchangeably herein, and refer to a mammal, including, but not limited to, primates, including simians and humans.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, murines, simians, humans, mammalian farm animals, mammalian sport animals, and mammalian pets.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a continuous delivery device (e.g., a pump or other controlled release injectible system); and a single subcutaneous injection followed by installation of a continuous delivery system.

“Continuous delivery” as used herein (e.g., in the context of “continuous delivery of a substance to a tissue”) is meant to refer to movement of drug to a delivery site, e.g., into a tissue in a fashion that provides for delivery of a desired amount of substance into the tissue over a selected period of time, where about the same quantity of drug is received by the patient each minute during the selected period of time.

“Controlled release” as used herein (e.g., in the context of “controlled drug release”) is meant to encompass release of substance (e.g., a Type I or Type III interferon receptor agonist, e.g., IFN-α) at a selected or otherwise controllable rate, interval, and/or amount, which is not substantially influenced by the environment of use. “Controlled release” thus encompasses, but is not necessarily limited to, substantially continuous delivery, and patterned delivery (e.g., intermittent delivery over a period of time that is interrupted by regular or irregular time intervals).

“Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “controlled drug delivery device” is meant to encompass any device wherein the release (e.g., rate, timing of release) of a drug or other desired substance contained therein is controlled by or determined by the device itself and not substantially influenced by the environment of use, or releasing at a rate that is reproducible within the environment of use.

By “substantially continuous” as used in, for example, the context of “substantially continuous infusion” or “substantially continuous delivery” is meant to refer to delivery of drug in a manner that is substantially uninterrupted for a pre-selected period of drug delivery, where the quantity of drug received by the patient during any 8 hour interval in the pre-selected period never falls to zero. Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

By “substantially steady state” as used in the context of a biological parameter that may vary as a function of time, it is meant that the biological parameter exhibits a substantially constant value over a time course, such that the area under the curve defined by the value of the biological parameter as a function of time for any 8 hour period during the time course (AUC8hr) is no more than about 20% above or about 20% below, and preferably no more than about 15% above or about 15% below, and more preferably no more than about 10% above or about 10% below, the average area under the curve of the biological parameter over an 8 hour period during the time course (AUC8hr average). The AUC8hr average is defined as the quotient (q) of the area under the curve of the biological parameter over the entirety of the time course (AUCtotal) divided by the number of 8 hour intervals in the time course (total/3 days), i.e., q=(AUCtotal)/(total/3 days). For example, in the context of a serum concentration of a drug, the serum concentration of the drug is maintained at a substantially steady state during a time course when the area under the curve of serum concentration of the drug over time for any 8 hour period during the time course (AUC8hr) is no more than about 20% above or about 20% below the average area under the curve of serum concentration of the drug over an 8 hour period in the time course (AUC8hr average), i.e., the AUC8hr is no more than 20% above or 20% below the AUC8hr average for the serum concentration of the drug over the time course.

The term “homologous” or “variants” as used herein in reference to proteins refers to a sequence similarity or identity, with identity being preferred. As is known in the art, a number of different programs can be used to identify whether a protein (or nucleic acid as discussed below) has sequence identity or similarity to a known sequence. Thus, in a preferred embodiment, homologous proteins or variants have an amino acid sequence that can differ from a wild-type sequence by up to about 40% of the residues, thus having about 60% homology. In other preferred embodiments, homologous proteins can have about 65, 70, 75, 80, 85, 90, 95, 96, 97, 98, or 99% homology.

The term “alkyl” used herein refers to a monovalent straight or branched chain radical of from one to twenty carbon atoms, including, but not limited to, methyl, ethyl, n-propyl, isopropyl, n-butyl, isobutyl, tert-butyl, n-hexyl, and the like.

The term “halo” used herein refers to fluoro, chloro, bromo, or iodo.

The term “alkoxy” used herein refers to straight or branched chain alkyl radical covalently bonded to the parent molecule through an —O— linkage. Examples of alkoxy groups include, but are not limited to, methoxy, ethoxy, propoxy, isopropoxy, butoxy, n-butoxy, sec-butoxy, t-butoxy and the like.

The term “alkenyl” used herein refers to a monovalent straight or branched chain radical of from two to twenty carbon atoms containing a carbon double bond including, but not limited to, 1-propenyl, 2-propenyl, 2-methyl-1-propenyl, 1-butenyl, 2-butenyl, and the like.

The term “alkynyl” used herein refers to a monovalent straight or branched chain radical of from two to twenty carbon atoms containing a carbon triple bond including, but not limited to, 1-propynyl, 1-butynyl, 2-butynyl, and the like.

The term “aryl” used herein refers to homocyclic aromatic radical whether fused or not fused. Examples of aryl groups include, but are not limited to, phenyl, naphthyl, biphenyl, phenanthrenyl, naphthacenyl, and the like.

The term “cycloalkyl” used herein refers to saturated aliphatic ring system radical having three to twenty carbon atoms including, but not limited to, cyclopropyl, cyclopentyl, cyclohexyl, cycloheptyl, and the like.

The term “cycloalkenyl” used herein refers to aliphatic ring system radical having three to twenty carbon atoms having at least one carbon-carbon double bond in the ring. Examples of cycloalkenyl groups include, but are not limited to, cyclopropenyl, cyclopentenyl, cyclohexenyl, cycloheptenyl, and the like.

The term “polycycloalkyl” used herein refers to saturated aliphatic ring system radical having at least two rings that are fused with or without bridgehead carbons. Examples of polycycloalkyl groups include, but are not limited to, bicyclo[4.4.0]decanyl, bicyclo[2.2.1]heptanyl, adamantyl, norbornyl, and the like.

The term “polycycloalkenyl” used herein refers to aliphatic ring system radical having at least two rings that are fused with or without bridgehead carbons in which at least one of the rings has a carbon-carbon double bond. Examples of polycycloalkenyl groups include, but are not limited to, norbornylenyl, 1,1′-bicyclopentenyl, and the like.

The term “polycyclic hydrocarbon” used herein refers to a ring system radical in which all of the ring members are carbon atoms. Polycyclic hydrocarbons can be aromatic or can contain less than the maximum number of non-cumulative double bonds. Examples of polycyclic hydrocarbon include, but are not limited to, naphthyl, dihydronaphthyl, indenyl, fluorenyl, and the like.

The term “heterocyclic” or “heterocyclyl” used herein refers to cyclic ring system radical having at least one ring system in which one or more ring atoms are not carbon, namely heteroatom. Heterocycles can be nonaromatic or aromatic. Examples of heterocyclic groups include, but are not limited to, morpholinyl, tetrahydrofuranyl, dioxolanyl, pyrrolidinyl, oxazolyl, pyranyl, pyridyl, pyrimidinyl, pyrrolyl, and the like.

The term “heteroaryl” used herein refers to heterocyclic group formally derived from an arene by replacement of one or more methine and/or vinylene groups by trivalent or divalent heteroatoms, respectively, in such a way as to maintain the aromatic system. Examples of heteroaryl groups include, but are not limited to, pyridyl, pyrrolyl, oxazolyl, indolyl, and the like.

The term “arylalkyl” used herein refers to one or more aryl groups appended to an alkyl radical. Examples of arylalkyl groups include, but are not limited to, benzyl, phenethyl, phenpropyl, phenbutyl, and the like.

The term “cycloalkylalkyl” used herein refers to one or more cycloalkyl groups appended to an alkyl radical. Examples of cycloalkylalkyl include, but are not limited to, cyclohexylmethyl, cyclohexylethyl, cyclopentylmethyl, cyclopentylethyl, and the like.

The term “heteroarylalkyl” used herein refers to one or more heteroaryl groups appended to an alkyl radical. Examples of heteroarylalkyl include, but are not limited to, pyridylmethyl, furanylmethyl, thiophenylethyl, and the like.

The term “heterocyclylalkyl” used herein refers to one or more heterocyclyl groups appended to an alkyl radical. Examples of heterocyclylalkyl include, but are not limited to, morpholinylmethyl, morpholinylethyl, morpholinylpropyl, tetrahydrofuranylmethyl, pyrrolidinylpropyl, and the like.

The term “aryloxy” used herein refers to an aryl radical covalently bonded to the parent molecule through an —O— linkage.

The term “alkylthio” used herein refers to straight or branched chain alkyl radical covalently bonded to the parent molecule through an —S— linkage.

The term “arylthio” used herein refers to an aryl radical covalently bonded to the parent molecule through an —S— linkage.

The term “alkylamino” used herein refers to nitrogen radical with one or more alkyl groups attached thereto. Thus, monoalkylamino refers to nitrogen radical with one alkyl group attached thereto and dialkylamino refers to nitrogen radical with two alkyl groups attached thereto.

The term “cyanoamino” used herein refers to nitrogen radical with nitrile group attached thereto.

The term “carbamyl” used herein refers to RNHCOO—.

The term “keto” and “carbonyl” used herein refers to C═O.

The term “carboxy” used herein refers to —COOH.

The term “sulfamyl” used herein refers to —SO₂NH₂.

The term “sulfonyl” used herein refers to —SO₂—.

The term “sulfinyl” used herein refers to —SO—.

The term “thiocarbonyl” used herein refers to C═S.

The term “thiocarboxy” used herein refers to CSOH.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

As used herein, a substituted group is derived from the unsubstituted parent structure in which there has been an exchange of one or more hydrogen atoms for another atom or group. When substituted, the substituent group(s) is (are) one or more group(s) individually and independently selected from C₁-C₂₀ alkyl, C₁-C₆ alkenyl, C₁-C₂₀ alkynyl, C₃-C₂₀ cycloalkyl, C₃-C₂₀ heterocycloalkyl (e.g., tetrahydrofuryl), aryl, heteroaryl, halo (e.g., chloro, bromo, iodo and fluoro), cyano, hydroxy, C₁-C₂₀ alkoxy, aryloxy, sulfhydryl (mercapto), C₁-C₂₀ alkylthio, arylthio, mono- and di-(C₁-C₂₀)alkyl amino, quaternary ammonium salts, amino(C₁-C₂₀)alkoxy, hydroxy(C₁-C₂₀)alkylamino, amino(C₁-C₂₀)alkylthio, cyanoamino, nitro, carbamyl, keto (oxy), carbonyl, carboxy, glycolyl, glycyl, hydrazino, guanyl, sulfamyl, sulfonyl, sulfinyl, thiocarbonyl, thiocarboxy, and combinations thereof. The protecting groups that can form the protective derivatives of the above substituents are known to those of skill in the art and can be found in references such as Greene and Wuts Protective Groups in Organic Synthesis; John Wiley and Sons: New York, 1999. Wherever a substituent is described as “optionally substituted” that substituent can be substituted with the above substituents.

Asymmetric carbon atoms may be present in the compounds described. All such isomers, including diastereomers and enantiomers, as well as the mixtures thereof are intended to be included in the scope of the recited compound. In certain cases, compounds can exist in tautomeric forms. All tautomeric forms are intended to be included in the scope of the recited compound. Likewise, when compounds contain an alkenyl or alkenylene group, there exists the possibility of cis- and trans-isomeric forms of the compounds. Both cis- and trans-isomers, as well as the mixtures of cis- and trans-isomers, are contemplated. Thus, reference herein to a compound includes all of the aforementioned isomeric forms unless the context clearly dictates otherwise.

Various forms are included in the embodiments, including polymorphs, solvates, hydrates, conformers, salts, and prodrug derivatives. A polymorph is a composition having the same chemical formula, but a different structure. A solvate is a composition formed by solvation (the combination of solvent molecules with molecules or ions of the solute). A hydrate is a compound formed by an incorporation of water. A conformer is a structure that is a conformational isomer. Conformational isomerism is the phenomenon of molecules with the same structural formula but different conformations (conformers) of atoms about a rotating bond. Salts of compounds can be prepared by methods known to those skilled in the art. For example, salts of compounds can be prepared by reacting the appropriate base or acid with a stoichiometric equivalent of the compound. A prodrug is a compound that undergoes biotransformation (chemical conversion) before exhibiting its pharmacological effects. For example, a prodrug can thus be viewed as a drug containing specialized protective groups used in a transient manner to alter or to eliminate undesirable properties in the parent molecule. Thus, reference herein to a compound includes all of the aforementioned forms unless the context clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range is encompassed within the embodiments. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the embodiments.

Unless defined otherwise, all technical and scientific terms used herein have the same meaning as commonly understood by one of ordinary skill in the art to which the embodiments belong. Although any methods and materials similar or equivalent to those described herein can also be used in the practice or testing of the embodiments, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The present embodiments provide compounds of Formulas I-IV, as well as pharmaceutical compositions and formulations comprising any compound of Formulas I-IV. A subject compound is useful for treating HCV infection and other disorders, as discussed below.

### Compositions

The present embodiments provide compounds having the general Formula I:

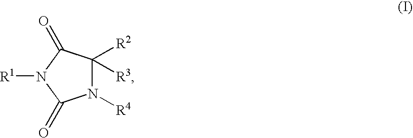

wherein:

R¹ is an optionally substituted aryl, an optionally substituted heterocyclyl comprising at least one of N, O or S, optionally substituted arylalkyl, or an optionally substituted heterocyclylalkyl comprising at least one of N, O or S in the heterocyclyl system;

R², R³ and R⁴ are each individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₆ to C₂₀ arylalkyl, optionally substituted C₃ to C₂₀ cycloalkylalkyl, optionally substituted C₅ to C₂₀ heteroarylalkyl, optionally substituted C₃ to C₂₀ heterocycylalkyl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; or

at least two of R², R³ and R⁴ join to form a ring wherein the ring is an unsubstituted or substituted 3 to 20 membered ring, wherein the members of the ring are selected from the group consisting of carbon, nitrogen, oxygen, and sulfur;

wherein formula (I) does not include the following structure:

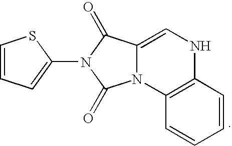

The present embodiments provide compounds having the general Formula II:

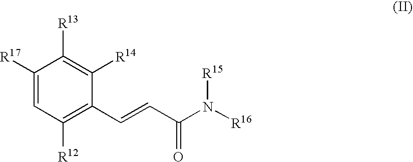

wherein:

R¹², R¹³, R¹⁴, and R¹⁷ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; wherein not all of R¹², R¹³, R¹⁴, and R¹⁷ are H;

R¹⁵ and R¹⁶ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₃ to C₂₀ heterocyclylalkyl, optionally substituted C₅ to C₂₀ heteroarylalkyl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, mono- and di-(C₁ to C₂₀)alkylamino, carbamyl, keto, carbonyl, carboxy, glycolyl, glycyl, hydrazino, guanylyl, and combinations thereof; or

R¹⁵ and R¹⁶ together form a ring wherein the ring is an unsubstituted or substituted 3 to 7 membered ring, wherein the members of the ring are selected from the group consisting of carbon, nitrogen, oxygen, or sulfur;

wherein formula (ID does not include the following structures:

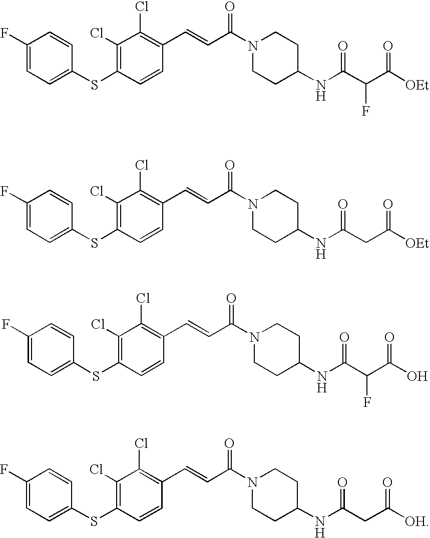

The present embodiments provide compounds having the general Formula III:

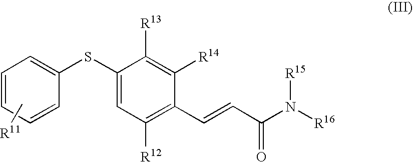

wherein:

R¹¹ is H, halo, optionally substituted C₁ to C₂₀ alkyl, optionally substituted C₁ to C₂₀ alkenyl, optionally substituted C₁ to C₂₀ alkynyl, or optionally substituted C₁ to C₂₀ alkoxy;

R¹², R¹³, and R¹⁴ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; wherein not all of R¹², R¹³, R¹⁴, and R¹⁷ are H;

R¹⁵ and R¹⁶ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₃ to C₂₀ heterocyclylalkyl, optionally substituted C₅ to C₂₀ heteroarylalkyl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, mono- and di-(C₁ to C₂₀)alkylamino, carbamyl, keto, carbonyl, carboxy, glycolyl, glycyl, hydrazino, guanylyl, and combinations thereof; or

R¹⁵ and R¹⁶ together form a ring wherein the ring is an unsubstituted or substituted 3 to 7 membered ring, wherein the members of the ring are selected from the group consisting of carbon, nitrogen, oxygen, or sulfur.

The present embodiments provide compounds having the general Formula IV:

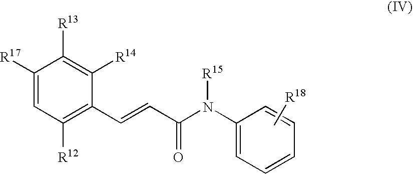

R¹³

R¹I R¹⁴

R¹⁵

Ni R¹⁸

R¹² 0

(I)

wherein:

R¹², R¹³, R¹⁴, and R¹⁷ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; wherein not all of R¹², R¹³, R¹⁴, and R¹⁷ are H;

R¹⁵ is selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₃ to C₂₀ heterocyclylalkyl, optionally substituted C₅ to C₂₀ heteroarylalkyl, optionally substituted C₁ to alkoxy, optionally substituted C₅ to C₂₀ aryloxy, optionally substituted C₁ to C₂₀ alkylthio, optionally substituted C₁ to C₂₀ arylthio, mono- and di-(C₁ to C₂₀)alkylamino, carbamyl, keto, carbonyl, carboxy, glycolyl, glycyl, hydrazino, guanylyl, and combinations thereof,

R¹⁸ is selected from the group consisting of H, optionally substituted C₁ to C₂₀ alkyl, optionally substituted C₁ to C₂₀ alkoxy, mono- and di-(C₁ to C₂₀)alkylamino, carbamyl, keto, carbonyl, carboxy, glycolyl, glycyl, hydrazino, guanylyl, and combinations thereof.

Examples of compounds of Formula I are set forth in Table 1 below.

TABLE 1

| | |
|---|---|
| 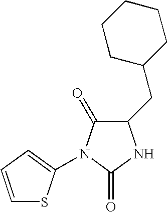 | I-1 |
| 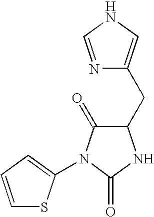 | I-2 |
| 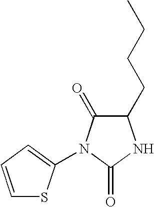 | I-3 |
| 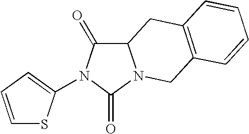 | I-4 |
| 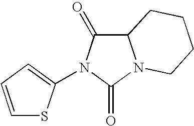 | I-5 |
| 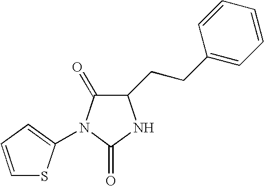 | I-6 |
| 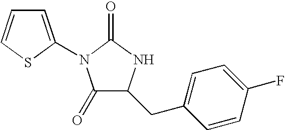 | I-7 |
| 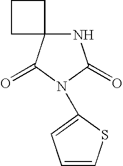 | I-8 |
| 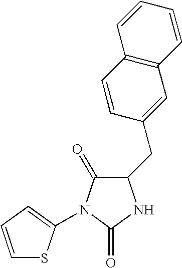 | I-9 |
| 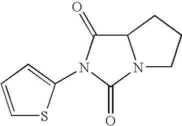 | I-10 |
| 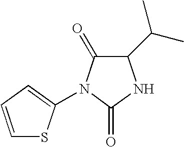 | I-11 |
| 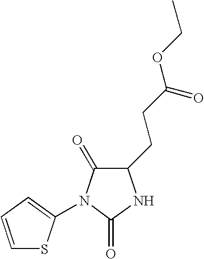 | I-12 |
| 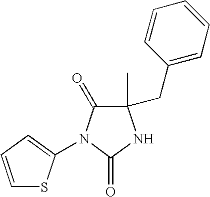 | I-13 |
| 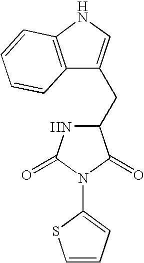 | I-14 |
| 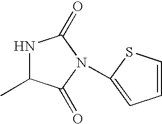 | I-15 |
| 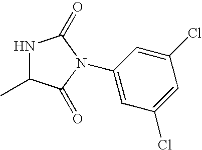 | I-16 |
| 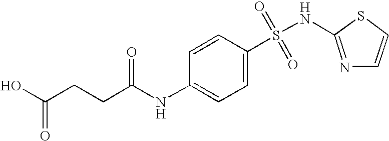 | I-17 |
| 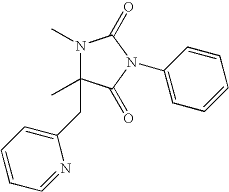 | I-18 |
| 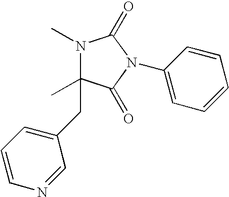 | I-19 |
| 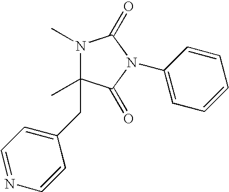 | I-20 |
| 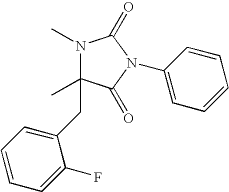 | I-21 |
| 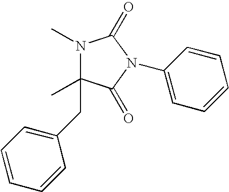 | I-22 |
| 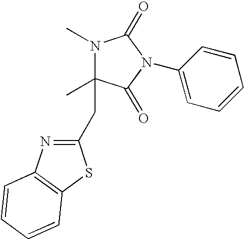 | I-23 |
| 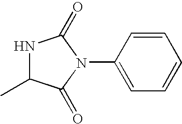 | I-24 |
| 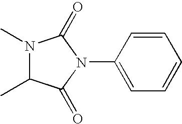 | I-25 |
| 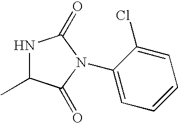 | I-26 |
| 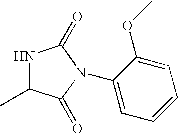 | I-27 |
| 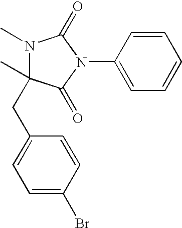 | I-28 |
| 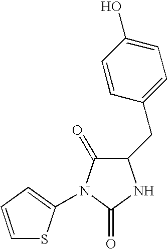 | I-29 |
| 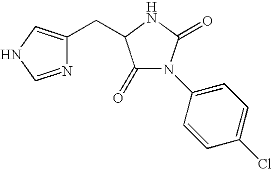 | I-30 |
| 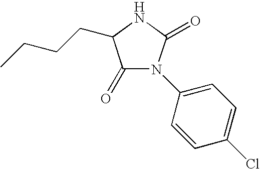 | I-31 |
| 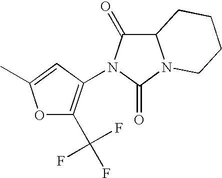 | I-32 |
| 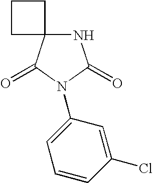 | I-33 |
| 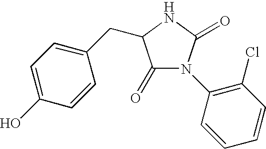 | I-34 |
| 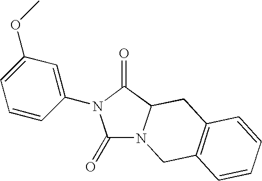 | I-35 |
| 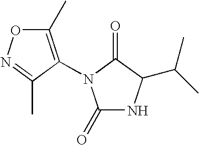 | I-36 |
| 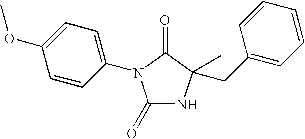 | I-37 |
| 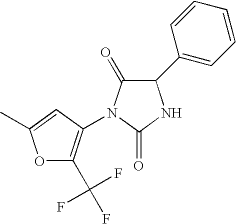 | I-38 |
| 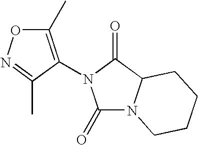 | I-39 |
| 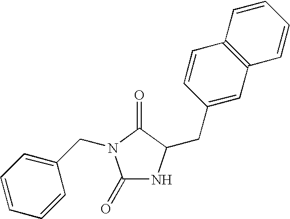 | I-40 |
| 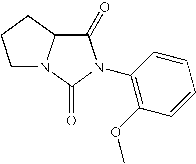 | I-41 |
| 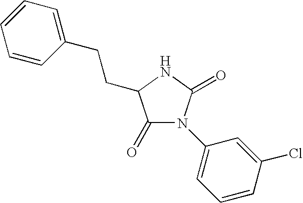 | I-42 |
| 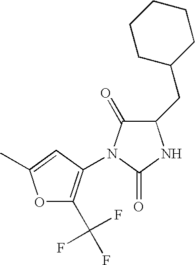 | I-43 |
| 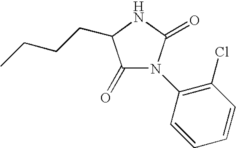 | I-44 |
| 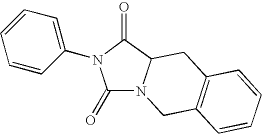 | I-45 |
| 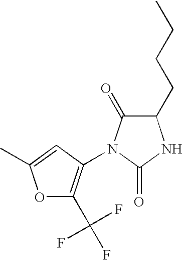 | I-46 |
| 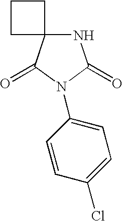 | I-47 |
| 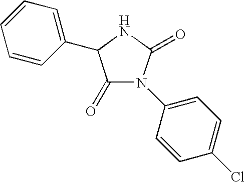 | I-48 |
| 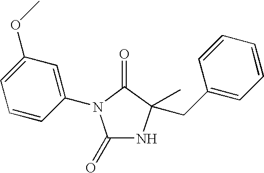 | I-49 |
| 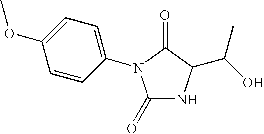 | I-50 |
| 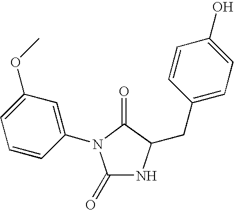 | I-51 |
| 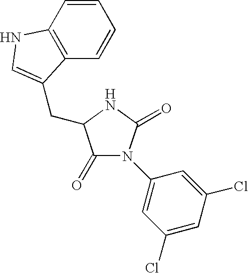 | I-52 |
| 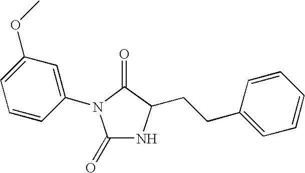 | I-53 |
| 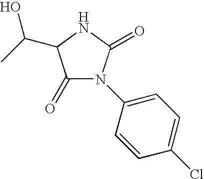 | I-54 |
| 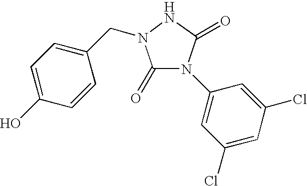 | I-55 |
| 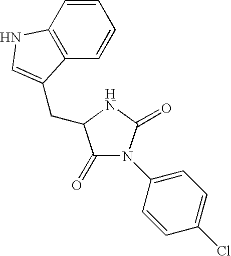 | I-56 |
| 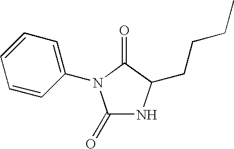 | I-57 |
| 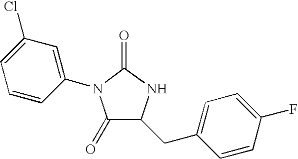 | I-58 |
| 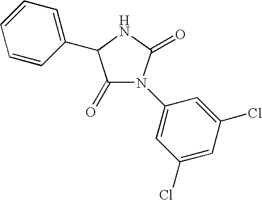 | I-59 |
| 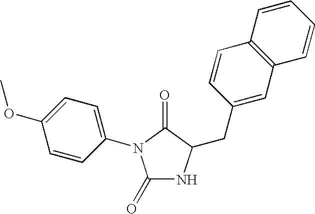 | I-60 |
| 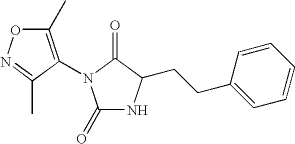 | I-61 |
| 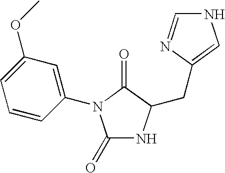 | I-62 |
| 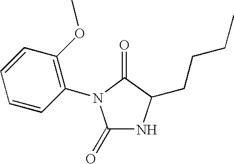 | I-63 |
| 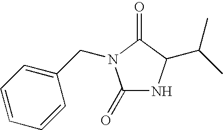 | I-64 |
| 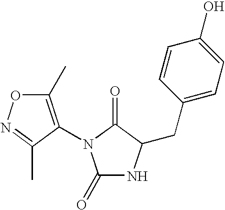 | I-65 |
| 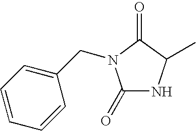 | I-66 |
| 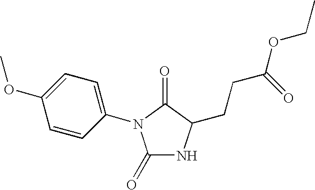 | I-67 |
| 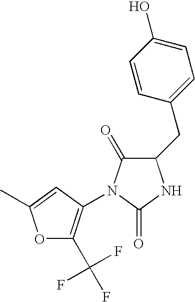 | I-68 |
| 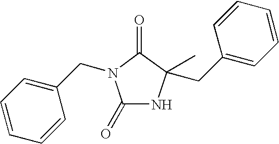 | I-69 |
| 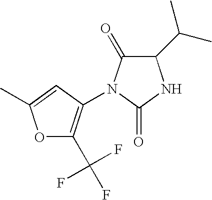 | I-70 |
| 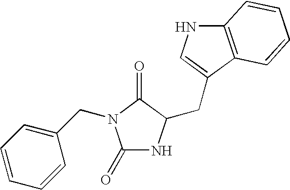 | I-71 |
| 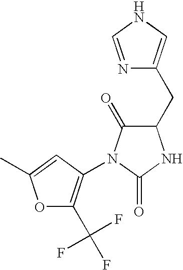 | I-72 |
| 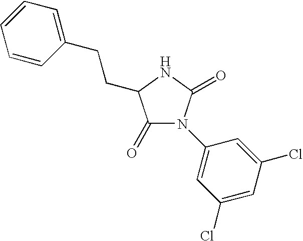 | I-73 |
| 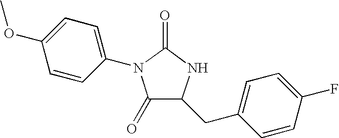 | I-74 |
| 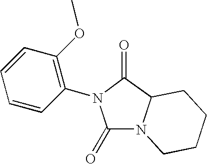 | I-75 |
| 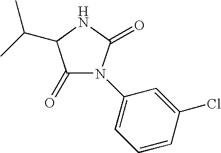 | I-76 |
| 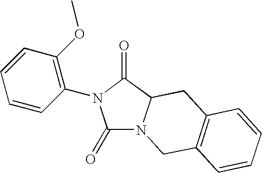 | I-77 |
| 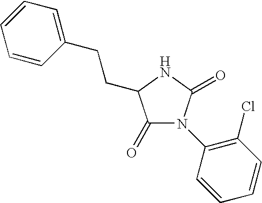 | I-78 |
| 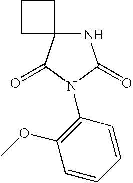 | I-79 |
| 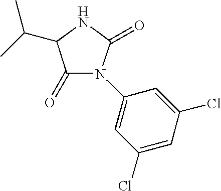 | I-80 |
| 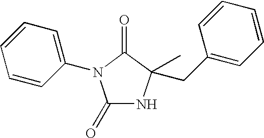 | I-81 |
| 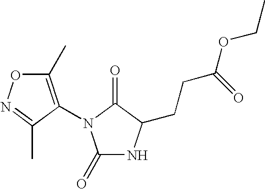 | I-82 |
| 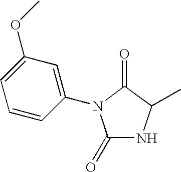 | I-83 |
| 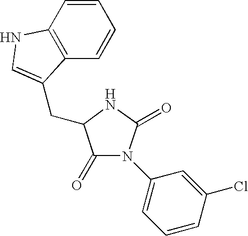 | I-84 |
| 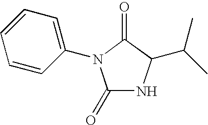 | I-85 |
| 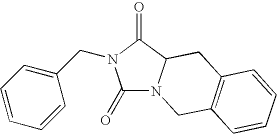 | I-86 |
| 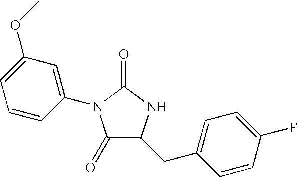 | I-87 |
| 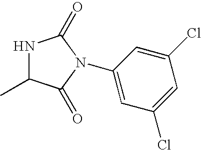 | I-88 |
| 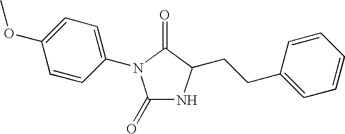 | I-89 |
| 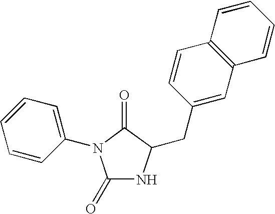 | I-90 |
| 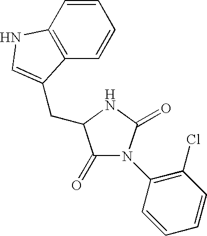 | I-91 |
| 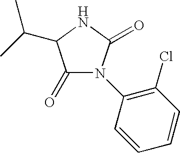 | I-92 |
| 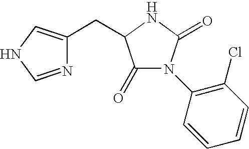 | I-93 |
| 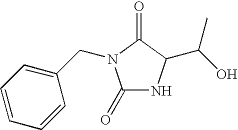 | I-94 |
| 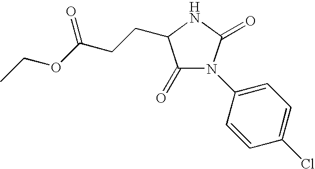 | I-95 |
| 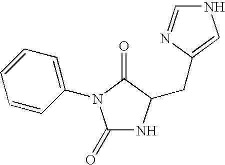 | I-96 |
| 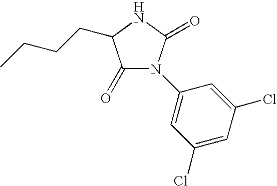 | I-97 |
| 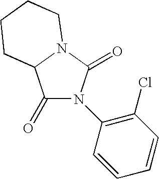 | I-98 |
| 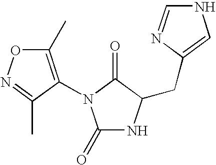 | I-99 |
| 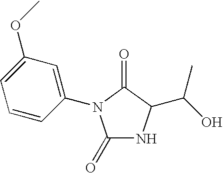 | I-100 |
| 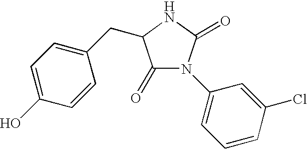 | I-101 |
| 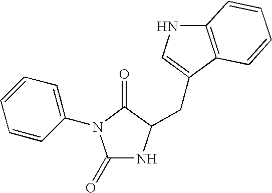 | I-102 |
| 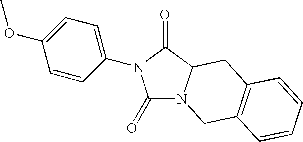 | I-103 |
| 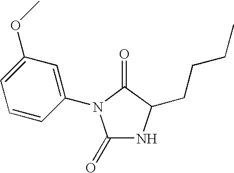 | I-104 |
| 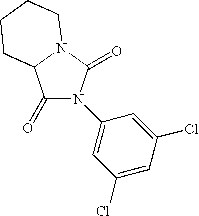 | I-105 |
| 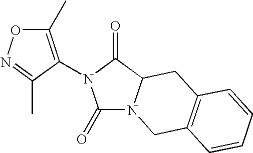 | I-106 |
| 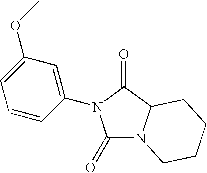 | I-107 |
| 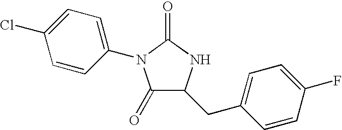 | I-108 |
| 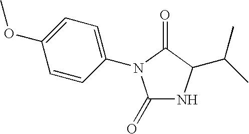 | I-109 |
| 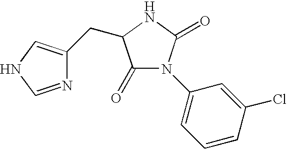 | I-110 |
| 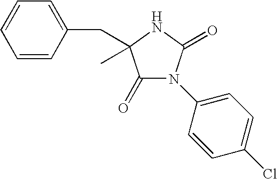 | I-111 |
| 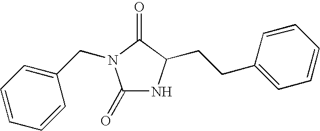 | I-112 |
| 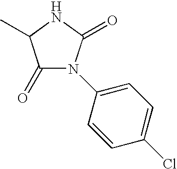 | I-113 |
| 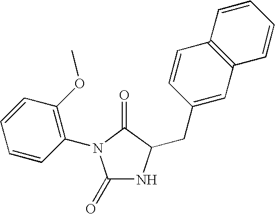 | I-114 |
| 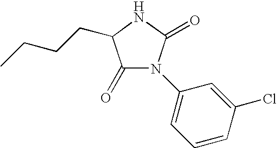 | I-115 |
| 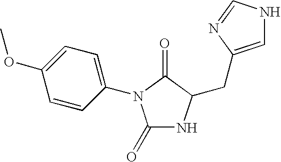 | I-116 |
| 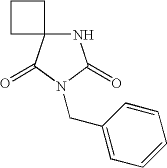 | I-117 |
| 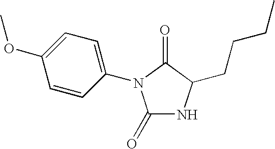 | I-118 |
| 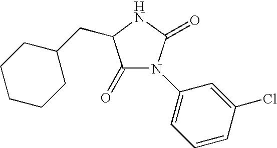 | I-119 |
| 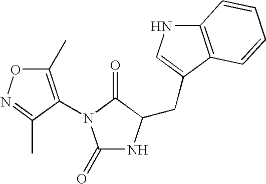 | I-120 |
| 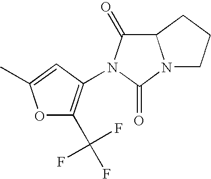 | I-121 |
| 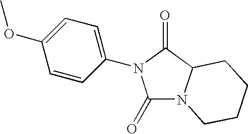 | I-122 |
| 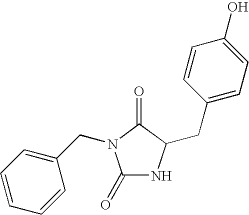 | I-123 |
| 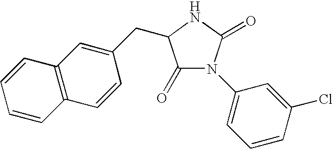 | I-124 |
| 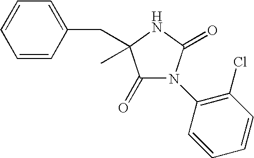 | I-125 |
| 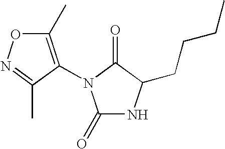 | I-126 |
| 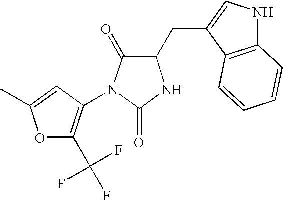 | I-127 |
| 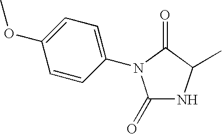 | I-128 |
| 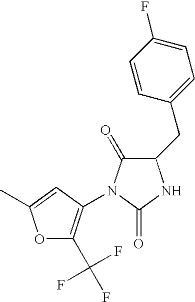 | I-129 |
| 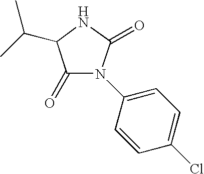 | I-130 |
| 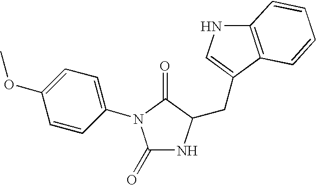 | I-131 |
| 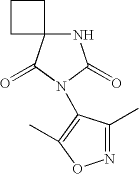 | I-132 |
| 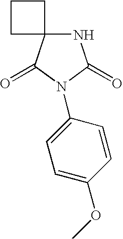 | I-133 |
| 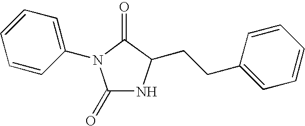 | I-134 |
| 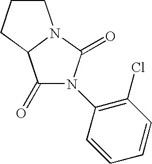 | I-135 |
| 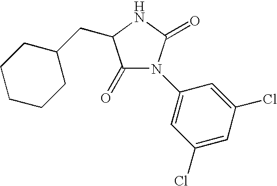 | I-136 |
| 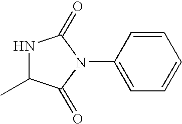 | I-137 |
| 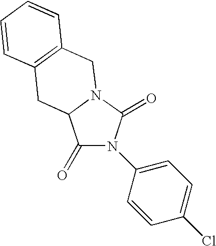 | I-138 |
| 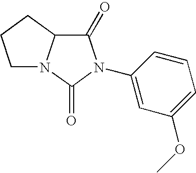 | I-139 |
| 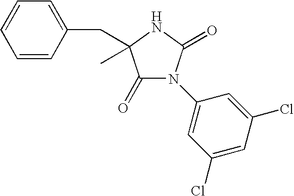 | I-140 |
| 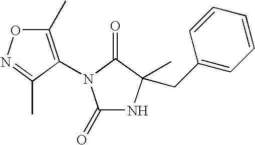 | I-141 |
| 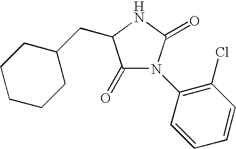 | I-142 |
| 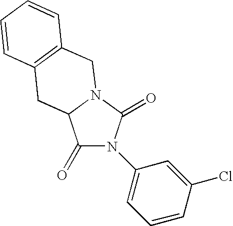 | I-143 |
| 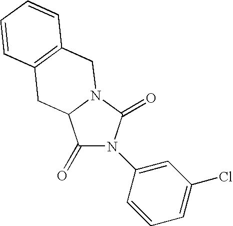 | I-144 |
| 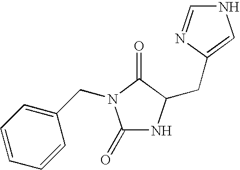 | I-145 |
| 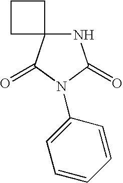 | I-146 |
| 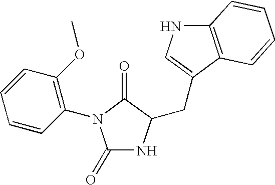 | I-147 |
| 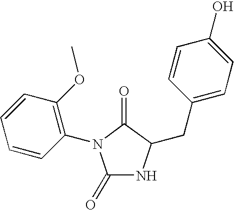 | I-148 |
| 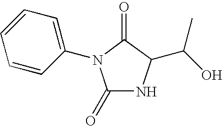 | I-149 |
| 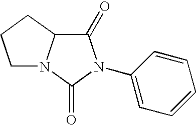 | I-150 |
| 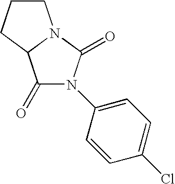 | I-151 |
| 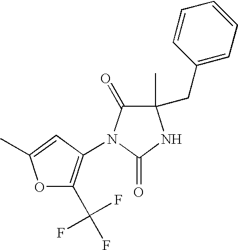 | I-152 |
| 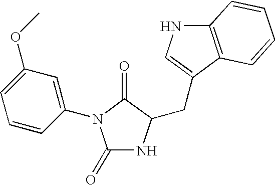 | I-153 |
| 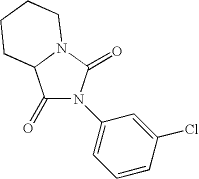 | I-154 |
| 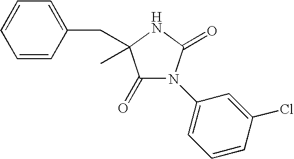 | I-155 |
| 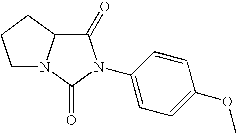 | I-156 |
| 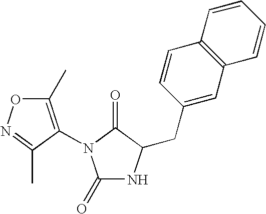 | I-157 |
| 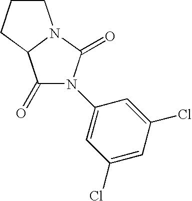 | I-158 |
| 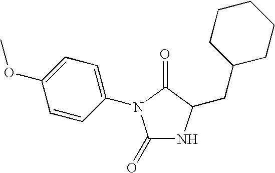 | I-159 |
| 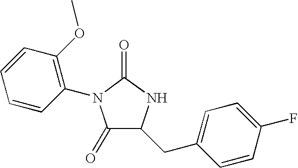 | I-160 |
| 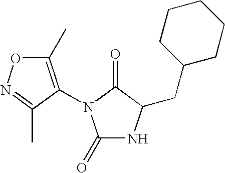 | I-161 |
| 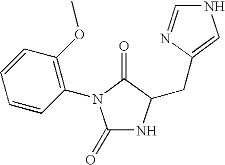 | I-162 |
| 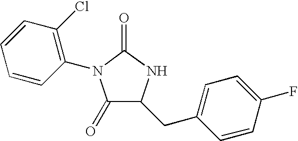 | I-163 |
| 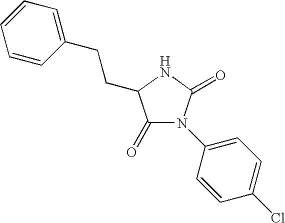 | I-164 |
| 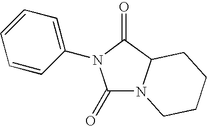 | I-165 |
| 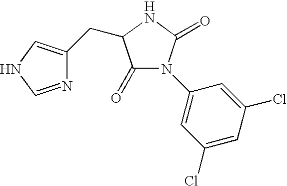 | I-166 |
| 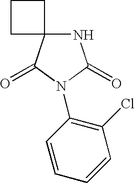 | I-167 |
| 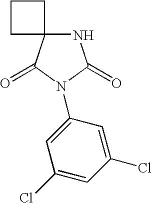 | I-168 |
| 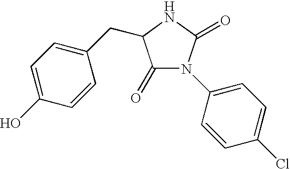 | I-169 |
| 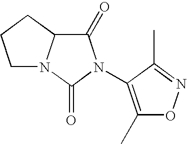 | I-170 |
| 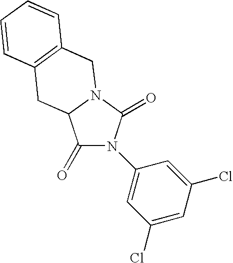 | I-171 |
| 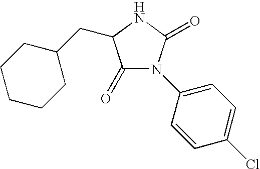 | I-172 |
| 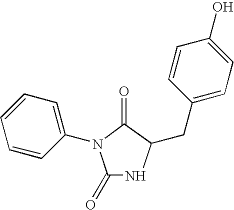 | I-173 |
| 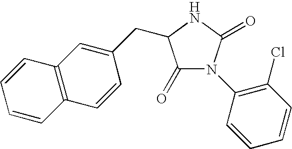 | I-174 |
| 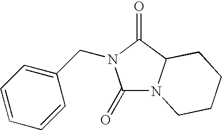 | I-175 |
| 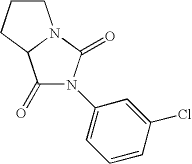 | I-176 |
| 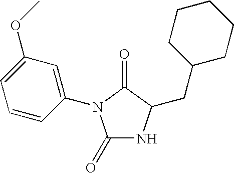 | I-177 |
| 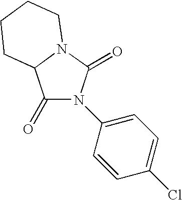 | I-178 |
| 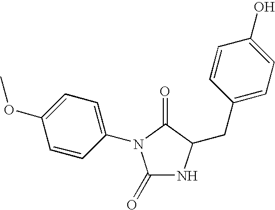 | I-179 |
| 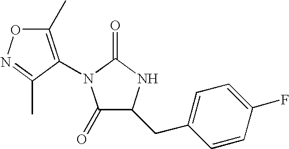 | I-180 |
| 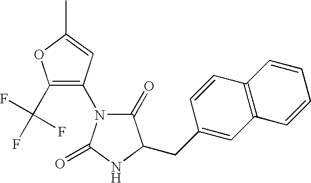 | I-181 |
| 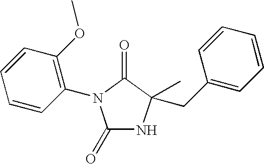 | I-182 |
| 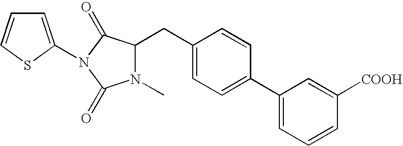 | I-183 |

Examples of compounds of Formulas II-IV are set forth in Table 2 below.

TABLE 2

| | |
|---|---|
| 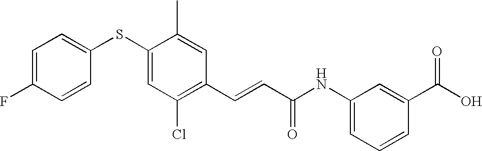 | II-1 |
| 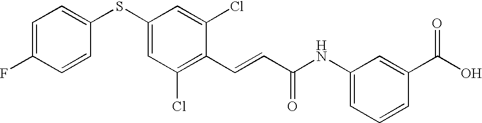 | II-2 |
| 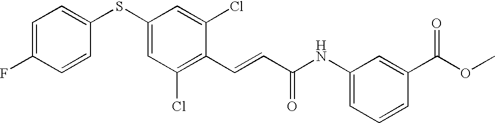 | II-3 |
| 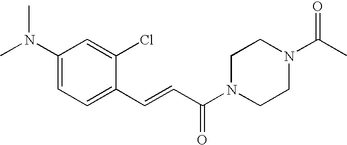 | II-4 |
| 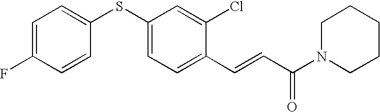 | II-5 |
| 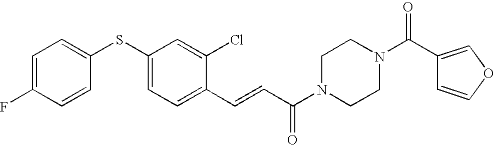 | II-6 |
| 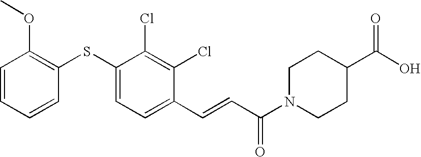 | II-7 |
| 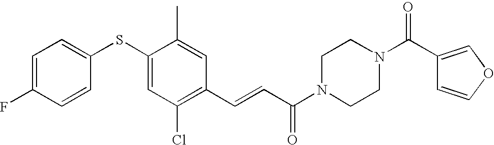 | II-8 |
| 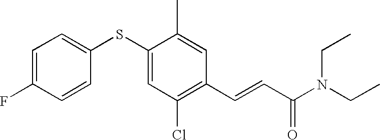 | II-9 |
| 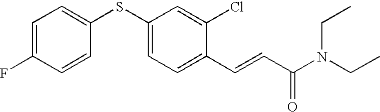 | II-10 |
| 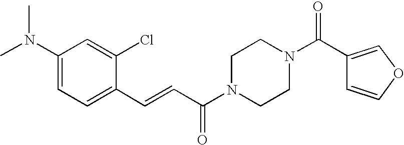 | II-11 |
| 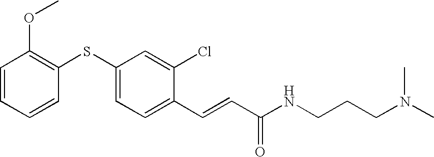 | II-12 |
| 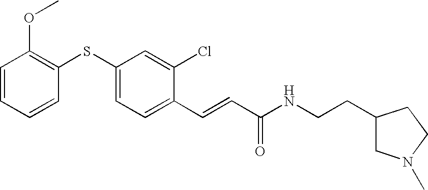 | II-13 |
| 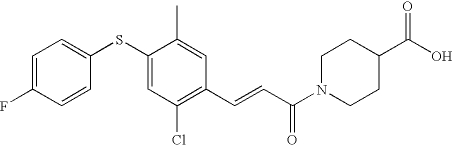 | II-14 |
| 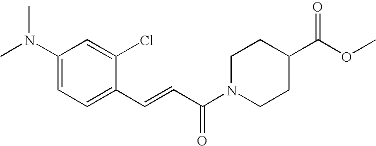 | II-15 |
| 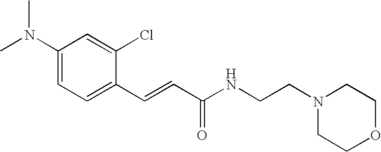 | II-16 |
| 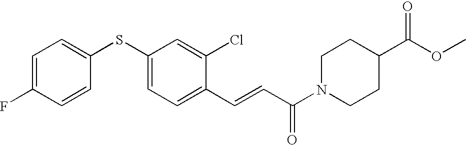 | II-17 |
| 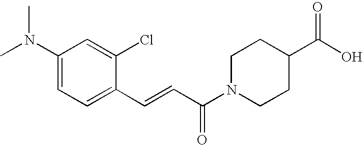 | II-18 |
| 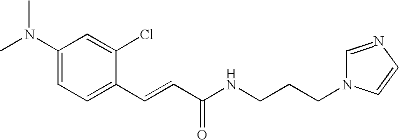 | II-19 |
| 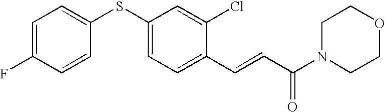 | II-20 |
| 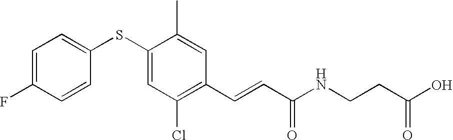 | II-21 |
| 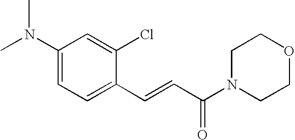 | II-22 |
| 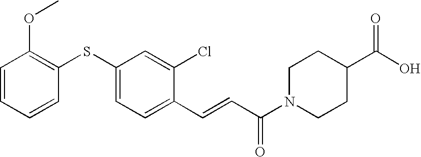 | II-23 |
| 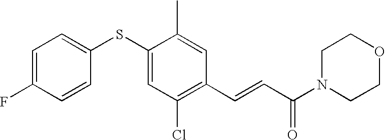 | II-24 |
| 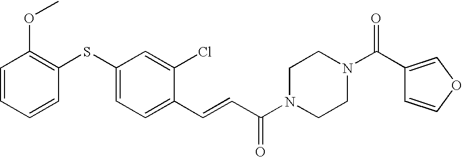 | II-25 |
| 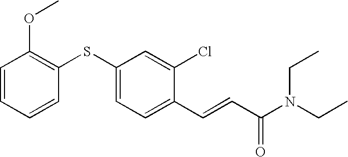 | II-26 |
| 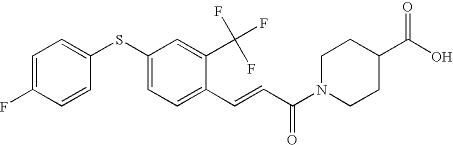 | II-27 |
| 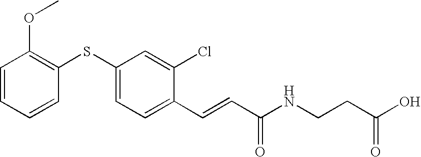 | II-28 |
| 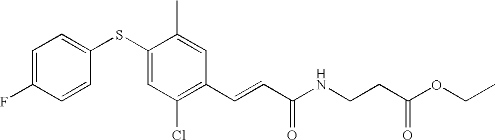 | II-29 |
| 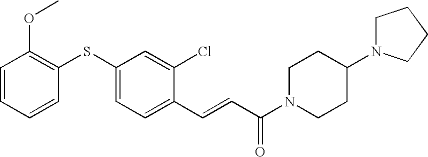 | II-30 |
| 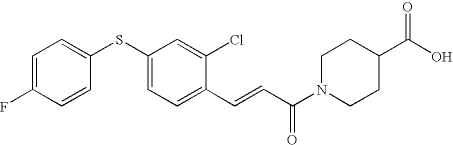 | II-31 |
| 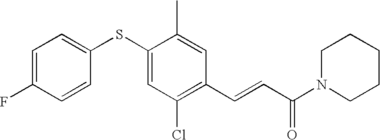 | II-32 |
| 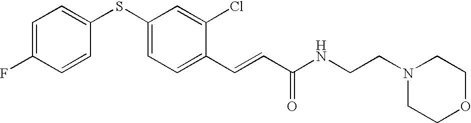 | II-33 |
| 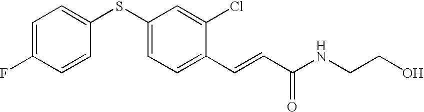 | II-34 |
| 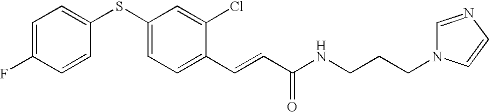 | II-35 |
| 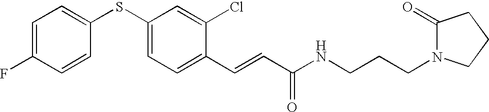 | II-36 |
| 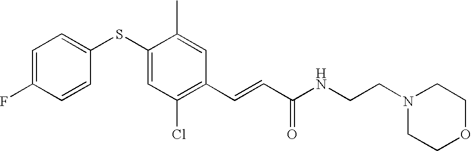 | II-37 |
| 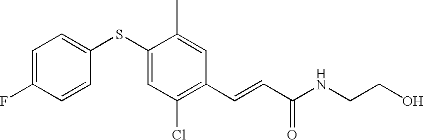 | II-38 |
| 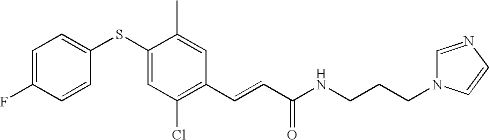 | II-39 |
| 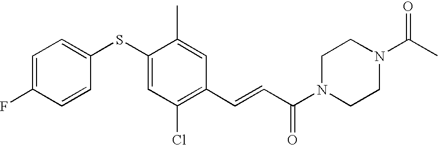 | II-40 |
| 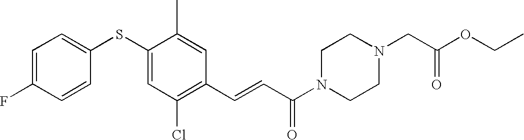 | II-41 |
| 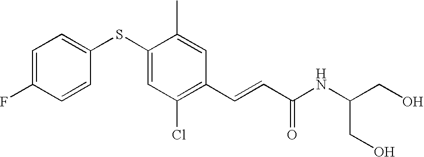 | II-42 |
| 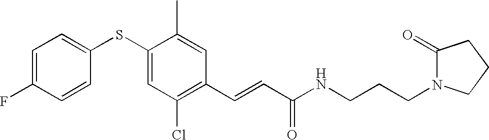 | II-43 |
| 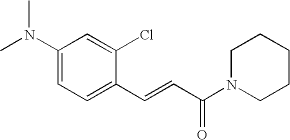 | II-44 |
| 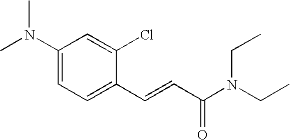 | II-45 |
| 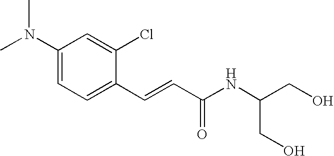 | II-46 |
| 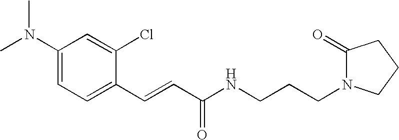 | II-47 |
| 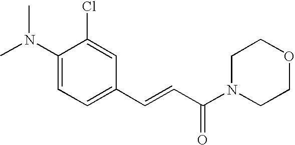 | II-48 |
| 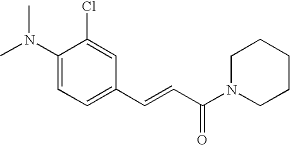 | II-49 |
| 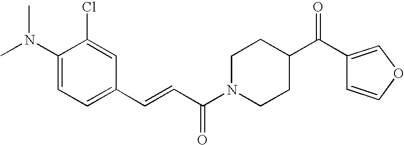 | II-50 |
| 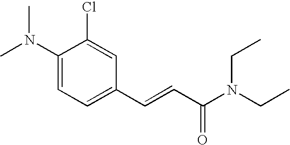 | II-51 |
| 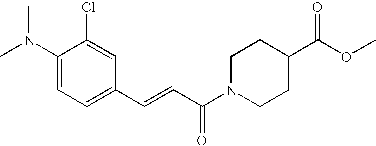 | II-52 |
| 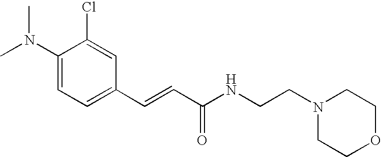 | II-53 |
| 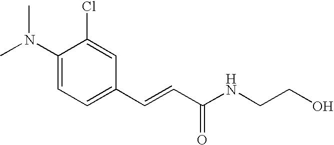 | II-54 |
| 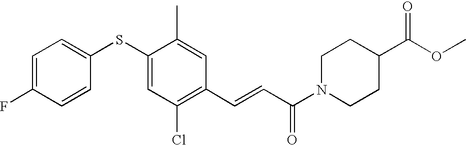 | II-55 |
| 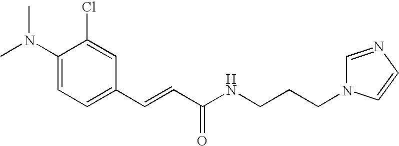 | II-56 |
| 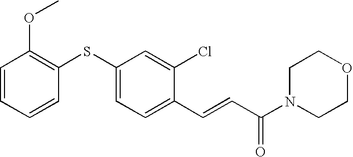 | II-57 |
| 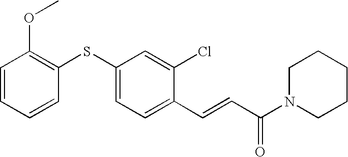 | II-58 |
| 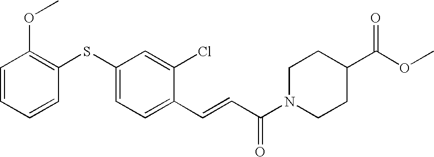 | II-59 |
| 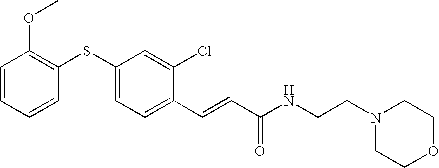 | II-60 |
| 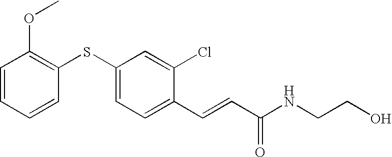 | II-61 |
| 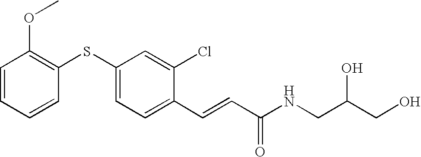 | II-62 |
| 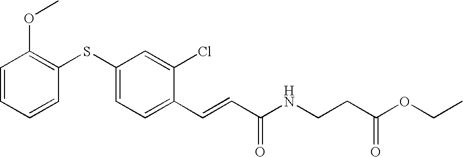 | II-63 |
| 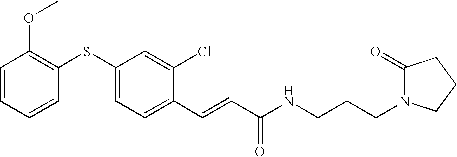 | II-64 |
| 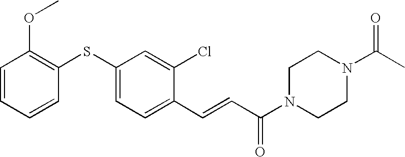 | II-65 |
| 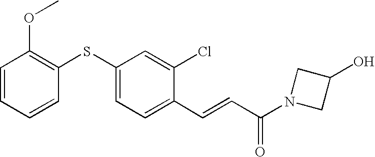 | II-66 |
| 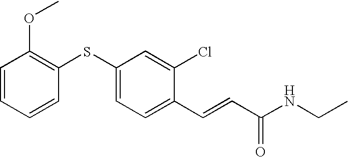 | II-67 |
| 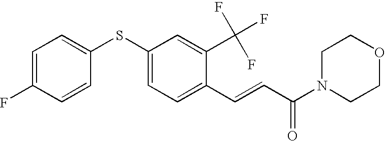 | II-68 |
| 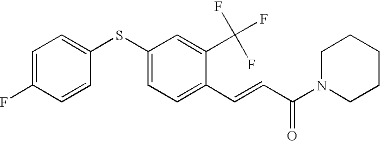 | II-69 |
| 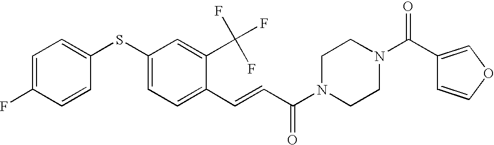 | II-70 |
| 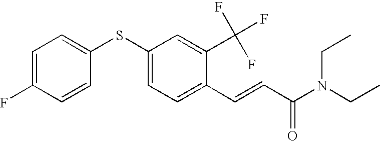 | II-71 |
| 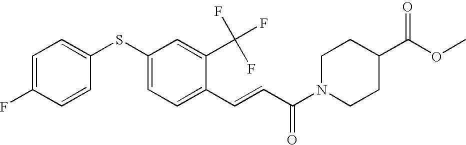 | II-72 |
| 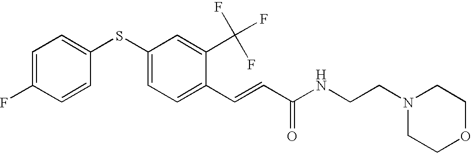 | II-73 |
| 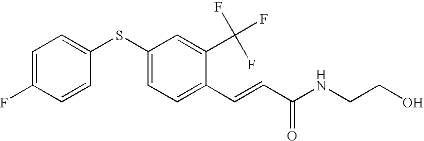 | II-74 |
| 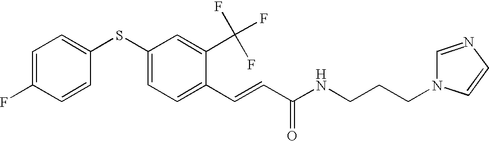 | II-75 |
| 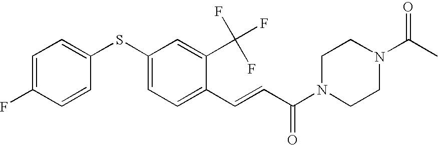 | II-76 |
| 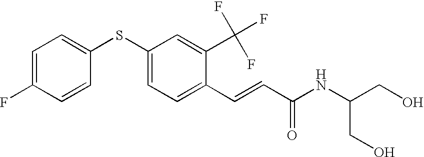 | II-78 |
| 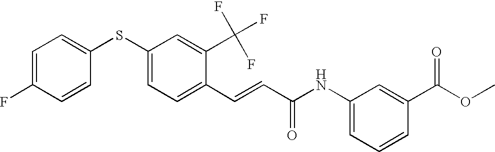 | II-79 |
| 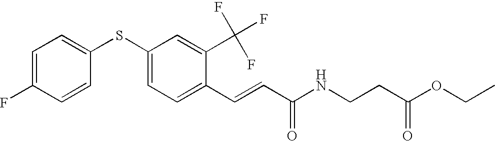 | II-80 |
| 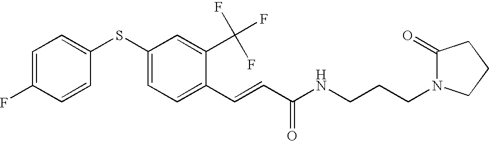 | II-81 |
| 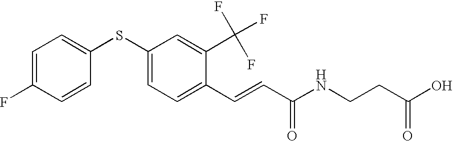 | II-82 |

In a preferred embodiment, there is provided a compound of the formula

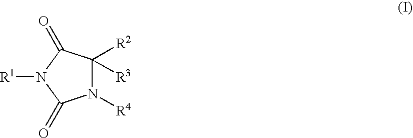

wherein:

R¹ is an optionally substituted aryl, an optionally substituted heterocyclyl comprising at least one of N, O or S, optionally substituted arylalkyl, or an optionally substituted heterocyclylalkyl comprising at least one of N, O or S in the heterocyclyl system;

R², R³ and R⁴ are each individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₆ to C₂₀ arylalkyl, optionally substituted C₃ to C₂₀ cycloalkylalkyl, optionally substituted C₅ to C₂₀ heteroarylalkyl, optionally substituted C₃ to C₂₀ heterocycylalkyl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; or

at least two of R², R³ and R⁴ join to form a ring wherein the ring is an unsubstituted or substituted 3 to 20 membered ring, wherein the members of the ring are selected from the group consisting of carbon, nitrogen, oxygen, and sulfur;

wherein formula (I) does not include the following structure:

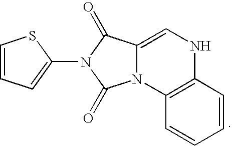

In a preferred embodiment, there is provided a compound of formula I, wherein R¹ is an optionally substituted aryl or an optionally substituted heterocyclyl comprising at least one of N, O or S.

In a preferred embodiment, there is provided a compound of formula I, wherein R², R³ and R⁴ are each individually selected from the group consisting of H, optionally substituted C₁ to C₂₀ alkyl, optionally substituted C₃ to C₂₀ partially saturated or fully saturated cycloalkyl, optionally substituted C₅ to C₂₀ aryl, optionally substituted C₆ to C₂₀ arylalkyl, optionally substituted C₃ to C₂₀ cycloalkylalkyl, optionally substituted C₅ to C₂₀ heteroarylalkyl, optionally substituted C₃ to C₂₀ heterocycylalkyl, optionally substituted C₁ to C₂₀ alkoxy, carbamyl, keto, carbonyl, carboxy, and combinations thereof.

In a preferred embodiment, there is provided a compound of formula I, wherein at least two of R², R³ and R⁴ join to form a ring wherein the ring is an unsubstituted or substituted 3 to 7 membered ring, wherein the members of the ring are selected from the group consisting of carbon, nitrogen, oxygen, or sulfur.

In a preferred embodiment, there is provided a compound of formula I, wherein R¹ is thiophene.

In a preferred embodiment, there is provided a compound of formula I, wherein R¹ is optionally substituted phenyl.

In a preferred embodiment, there is provided a compound of formula I, wherein R¹ is thiophene or optionally substituted phenyl, and wherein R², R³ and R⁴ are each individually selected from the group consisting of H, optionally substituted C₁ to C₂₀ alkyl, optionally substituted C₃ to C₂₀ partially saturated or fully saturated cycloalkyl, optionally substituted C₅ to C₂₀ aryl, optionally substituted C₆ to C₂₀ arylalkyl, optionally substituted C₃ to C₂₀ cycloalkylalkyl, optionally substituted C₅ to C₂₀ heteroarylalkyl, optionally substituted C₃ to C₂₀ heterocycylalkyl, optionally substituted C₁ to C₂₀ alkoxy, carbamyl, keto, carbonyl, carboxy, and combinations thereof.

In a preferred embodiment, there is provided a compound of formula I, wherein R¹ is thiophene or optionally substituted phenyl, and wherein at least two of R², R³ and R⁴ join to form a ring wherein the ring is an unsubstituted or substituted 3 to 7 membered ring, wherein the members of the ring are selected from the group consisting of carbon, nitrogen, oxygen, or sulfur.

An embodiment provides compounds of the Formulae (Ia), (Ib) and (Ic):

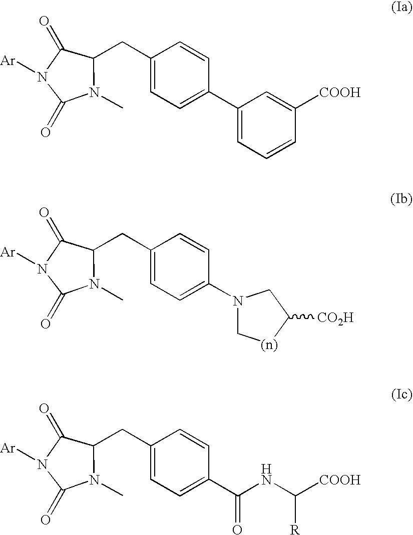

In Formulae (Ia), (Ib) and (Ic), Ar represents aryl (e.g., phenyl, thiophenyl, etc.) and n is one or two, representing the number of carbon atoms in the ring at the indicated position. For example, for n=1, the ring contains four carbon atoms and one nitrogen atom; for n=2, the ring contains five carbon atoms and one nitrogen atom. Compounds of the Formulae (Ia), (Ib) and (Ic) are examples of compounds of the Formula (I).

In a preferred embodiment, there is provided a compound of the formula I:

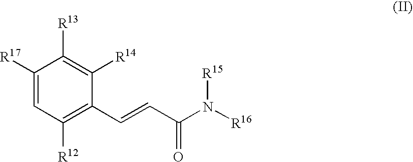

wherein:

R¹², R¹³, R¹⁴, and R¹⁷ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; wherein not all of R¹², R¹³, R¹⁴, and R¹⁷ are H;

R¹⁵ and R¹⁶ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₃ to C₂₀ heterocyclylalkyl, optionally substituted C₅ to C₂₀ heteroarylalkyl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, mono- and di-(C₁ to C₂₀)alkylamino, carbamyl, keto, carbonyl, carboxy, glycolyl, glycyl, hydrazino, guanylyl, and combinations thereof; or

R¹⁵ and R¹⁶ together form a ring wherein the ring is an unsubstituted or substituted 3 to 7 membered ring, wherein the members of the ring are selected from the group consisting of carbon, nitrogen, oxygen, or sulfur;

wherein formula (II) does not include the following structures:

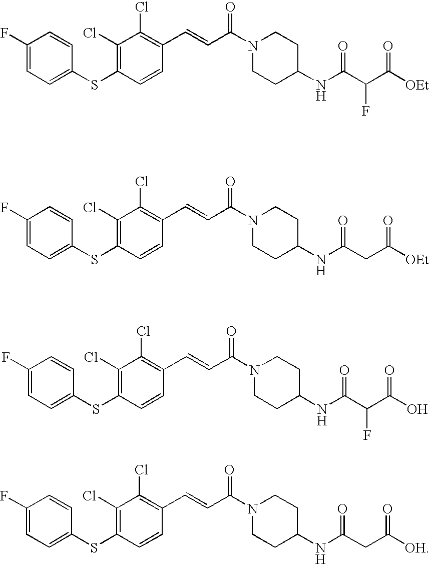

In a preferred embodiment, there is provided a compound of formula II, wherein R¹², R¹³, R¹⁴, and R¹⁷ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₁ to C₂₀ alkoxy, optionally substituted C₁ to C₂₀ alkylthio, halo, cyano, mercapto, hydroxy, mono- and di-(C₁ to C₂₀)alkylamino, cyanoamino, nitro, carbamyl, keto, carbonyl, and carboxy.

In a preferred embodiment, there is provided a compound of formula II, wherein R¹⁵ and R¹⁶ are individually selected from the group consisting of H, optionally substituted C₁ to C₂₀ alkyl, optionally substituted C₁ to C₂₀ alkenyl, optionally substituted C₁ to C₂₀ alkynyl, mono- and di-(C₁ to C₂₀)alkylamino, optionally substituted C₅ to C₂₀ aryl, optionally substituted C₃ to C₂₀ heterocyclylalkyl, optionally substituted C₅ to C₂₀ heteroarylalkyl, carbamyl, keto, carbonyl, carboxy, and combinations thereof.

In a preferred embodiment, there is provided a compound of formula II, wherein R¹⁵ and R¹⁶ together form a ring wherein the ring is an unsubstituted or substituted 4 to 6 membered ring, wherein the members of the ring are selected from the group consisting of carbon, nitrogen, oxygen, and sulfur.

In a preferred embodiment, there is provided a compound of formula II having the formula:

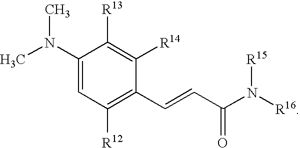

The present embodiments provide compounds having the general Formula III:

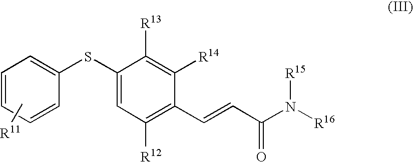

wherein:

R¹¹ is H, halo, optionally substituted C₁ to C₂₀ alkyl, optionally substituted C₁ to C₂₀ alkenyl, optionally substituted C₁ to C₂₀ alkynyl, or optionally substituted C₁ to C₂₀ alkoxy;

R¹², R¹³, and R¹⁴ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; wherein not all of R¹², R¹³, R¹⁴, and R¹⁷ are H;

R¹⁵ and R¹⁶ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₃ to C₂₀ heterocyclylalkyl, optionally substituted C₅ to C₂₀ heteroarylalkyl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, mono- and di-(C₁ to C₂₀)alkylamino, carbamyl, keto, carbonyl, carboxy, glycolyl, glycyl, hydrazino, guanylyl, and combinations thereof; or

R¹⁵ and R¹⁶ together form a ring wherein the ring is an unsubstituted or substituted 3 to 7 membered ring, wherein the members of the ring are selected from the group consisting of carbon, nitrogen, oxygen, or sulfur.

In a preferred embodiment, there is provided a compound of formula III, wherein R¹¹ is H, halo, optionally substituted C₁ to C₂₀ alkyl, or optionally substituted C₁ to C₂₀ alkoxy.

In a preferred embodiment, there is provided a compound of formula III, wherein R¹², R¹³, and R¹⁴ are individually selected from the group consisting of H, optionally substituted C₁ to C₂₀ alkyl, optionally substituted C₁ to C₂₀ alkoxy, optionally substituted C₁ to C₂₀ alkylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; wherein not all of R¹², R¹³, and R¹⁴ are H.

In a preferred embodiment, there is provided a compound of formula III, wherein R¹², R¹³, and R¹⁴ are individually selected from the group consisting of H, optionally substituted C₁ to C₂₀ alkyl, optionally substituted C₁ to C₂₀ alkoxy, optionally substituted C₁ to C₂₀ alkylthio, halo, hydroxy, mono- and di-(C₁ to C₂₀)alkylamino, and combinations thereof; wherein not all of R¹², R¹³, and R¹⁴ are H.

In a preferred embodiment, there is provided a compound of formula III, wherein R¹⁵ and R¹⁶ are individually selected from the group consisting of H, optionally substituted C₁ to C₂₀ alkyl, optionally substituted C₃ to C₂₀ partially saturated or fully saturated cycloalkyl, optionally substituted C₂ to C₂₀ partially saturated or fully saturated heterocyclic, optionally substituted C₅ to C₂₀ aryl, optionally substituted C₂ to C₂₀ heteroaryl, optionally substituted C₃ to C₂₀ heterocyclylalkyl, optionally substituted C₅ to C₂₀ heteroarylalkyl, optionally substituted C₁ to C₂₀ alkoxy, mono- and di-(C₁ to C₂₀)alkylamino, carbamyl, keto, carbonyl, carboxy, and combinations thereof.

In a preferred embodiment, there is provided a compound of formula III, wherein R¹⁵ and R¹⁶ together form a ring wherein the ring is an unsubstituted or substituted 4 or 6 membered ring, wherein the members of the ring are selected from the group consisting of carbon, nitrogen, oxygen, or sulfur.

In a preferred embodiment, there is provided a compound of formula III, wherein R¹¹ is fluoro and R¹², R¹³, and R¹⁴ are individually selected from the group consisting of H, alkyl, and halo.

The present embodiments provide compounds having the general Formula IV:

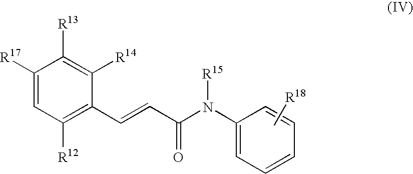

wherein:

R¹², R¹³, R¹⁴, and R¹⁷ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; wherein not all of R¹², R¹³, R¹⁴, and R¹⁷ are H;

R¹⁵ is selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₃ to C₂₀ heterocyclylalkyl, optionally substituted C₅ to C₂₀ heteroarylalkyl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, mono- and di-(C₁ to C₂₀)alkylamino, carbamyl, keto, carbonyl, carboxy, glycolyl, glycyl, hydrazino, guanylyl, and combinations thereof;

R¹⁸ is selected from the group consisting of H, optionally substituted C₁ to C₂₀ alkyl, optionally substituted C₁ to C₂₀ alkoxy, mono- and di-(C₁ to C₂₀)alkylamino, carbamyl, keto, carbonyl, carboxy, glycolyl, glycyl, hydrazino, guanylyl, and combinations thereof.

Preferred embodiments provide a method of treating a hepatitis C virus infection in an individual, the method comprising administering to the individual an effective amount of a composition comprising a preferred compound.

Preferred embodiments provide a method of treating liver fibrosis in an individual, the method comprising administering to the individual an effective amount of a composition comprising a preferred compound.

Preferred embodiments provide a method of increasing liver function in an individual having a hepatitis C virus infection, the method comprising administering to the individual an effective amount of a composition comprising a preferred compound.

The present embodiments further provide compositions, including pharmaceutical compositions, comprising compounds of the general Formulas I-IV, and salts, esters, or other derivatives thereof. A subject pharmaceutical composition comprises a subject compound; and a pharmaceutically acceptable excipient. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In many embodiments, a subject compound inhibits enzymatic activity of an HCV NS3 helicase with an IC₅₀ of less than about 50 μM, e.g., a subject compound inhibits an HCV NS3 protease with an IC₅₀ of less than about 40 μM, less than about 25 μM, less than about 10 μM, less than about 1 μM, less than about 100 nM, less than about 80 nM, less than about 60 nM, less than about 50 nM, less than about 25 nM, less than about 10 nM, or less than about 1 nM, or less.

In many embodiments, a subject compound inhibits HCV viral replication. For example, a subject compound inhibits HCV viral replication by at least about 10%, at least about 15%, at least about 20%, at least about 25%, at least about 30%, at least about 40%, at least about 50%, at least about 60%, at least about 70%, at least about 80%, or at least about 90%, or more, compared to HCV viral replication in the absence of the compound. Whether a subject compound inhibits HCV viral replication can be determined using methods known in the art, including an in vitro viral replication assay.

### NS3 Helicase

HCV is a positive strand RNA virus. Upon infection, its genomic RNA produces a large polyprotein that is processed by viral and cellular proteins into at least 10 different viral proteins. Like other positive strand RNA viruses, replication of the positive strand involves initial synthesis of a negative strand RNA. This negative strand RNA, which is a replication intermediate, serves as a template for the production of progeny genomic RNA. This process is believed to be carried out by two or more viral encoded enzymes, including RNA-dependent RNA polymerase and RNA helicase. RNA polymerase copies template RNA for the production of progeny RNA. This enzyme does not synthesize RNA molecules from DNA template.

The RNA helicase unwinds the secondary structure present in the single-strand RNA molecule. The helicase also unwinds the duplex RNA into single-strand forms. Genomic HCV RNA molecules contain extensive secondary structure. Replication intermediates of HCV RNA are believed to be present as duplex RNA consisting of positive and negative strand RNA molecules. The activity of RNA helicase is believed to facilitate the activity of RNA dependent RNA polymerase which is believed to unwind single stranded RNA molecules as a template. Therefore, the biological activity of helicase is believed to be important for HCV replication.

### Modulation of NS3 Helicase Activity

The NS3 helicase comprises about 631 amino acids (SEQ ID NO:1) including three domains: Domain 1, Domain 2, and Domain 3. Homologous structures of NS3 helicase are contemplated as part of the embodiments. Domain 1 comprises a region of residues or variants thereof extending from Residue 190 to Residue 324 as indicated in SEQ ID NO:1. Domain 2 comprises a region of residues or variants thereof extending from Residue 328 to Residue 483 as indicated in SEQ ID NO:1. Domains 1 and 2 form parallel β-sheets surrounded by α-helices.

It is believed that the compounds described herein (e.g., Formulas I-IV) bind to NS3 helicase at Domain 1 and/or Domain 2. Binding of the compounds to NS3 helicase on Domain 1 is believed to comprise interactions with one or more of Residues 209 to 221; Residues 286 to 288; Residues 317 to 319, and/or Residues 214 to 218 as indicated in SEQ ID NO:1.

Binding of the compounds to NS3 helicase on Domain 2 is believed to comprise interactions with one or more of Residues 412 to 423; Residue 363; Residue 365; Residue 406; Residue 408; Residue 391; Residue 397; Residue 400; and Residues 400 to 404 as indicated in SEQ ID NO:1.

Binding of a compound to NS3 helicase at Domain 1 and/or Domain 2 as described above may provoke movement of one or more of Residues 412 to 423. Additional movements of NS3 helicase may also occur. The movement or movements resulting from the binding of the compound may cause an allosteric movement of NS3 helicase such that binding of a nucleic acid substrate at a remote portion of the NS3 helicase may be inhibited. In preferred embodiments, the nucleic acid substrate is DNA or RNA. By inhibiting nucleic acid substrate binding, the activity of NS3 helicase may be modulated. In preferred embodiments, the modulation of NS3 helicase activity is inhibition of NS3 helicase activity. In preferred embodiments, the NS3 helicase activity that is modulated is replication of HCV. The modulation of NS3 helicase activity can occur in vivo or ex vivo.

An embodiment provides a compound comprising at least one functional group configured to facilitate binding of the compound to NS3 helicase, the binding being effective to modulate (e.g., inhibit) NS3 helicase activity. The compounds of Formulas I-IV are examples of compounds comprising such configured functional groups. For example, the compound may be any one or more of I-1 to I-183 or II-1 to II-82 described in Tables 1 and 2 above. In an embodiment, the binding is effective to inhibit unwinding of a nucleic acid substrate (e.g., DNA and/or RNA) by the NS3 helicase. The binding may facilitate allosteric movement of the NS3 helicase, thereby modulating NS3 helicase activity. The functional group may be configured to facilitate binding of the compound to NS3 helicase Domain 1, e.g., to one or more residues in NS3 helicase Domain 1. For example, the residue may be any one of Residues 209 to 221, Residues 286 to 288, Residues 317 to 319, or Residues 214 to 218. In another embodiment, the functional group may be configured to facilitate binding of the compound to NS3 helicase Domain 2, e.g., to one or more residues in NS3 helicase Domain 2. For example, the residue may be any one of Residues 412 to 423, Residue 363, Residue 365, Residue 406, Residue 408, Residue 391, Residue 397, Residue 400, or Residues 400 to 404.

Another embodiment provides a pharmaceutical composition that comprises a compound and a pharmaceutically acceptable carrier, wherein the compound comprises at least one functional group configured to facilitate binding of the compound to NS3 helicase, the binding being effective to modulate NS3 helicase activity, as described above. For example, the compound in the composition may be a compound of Formulas I-IV, and thus may be any one or more of compounds I-1 to I-183 or II-1 to II-82 described in Tables 1 and 2 above.

Another embodiment provides a method of modulating NS3 helicase activity comprising contacting an NS3 protein with a compound or a composition that comprises the compound, wherein the compound comprises at least one functional group configured to facilitate binding of the compound to NS3 helicase, the binding being effective to modulate NS3 helicase activity, as described above. The contacting may occur ex vivo or in vivo. If in vivo, the contacting may occur in a human body. In an embodiment, the method comprises identifying a person having a medical condition or disease as disclosed herein, e.g., a liver disease or condition such as HCV.

### Treating a Hepatitis Virus Infection

The methods and compositions described herein are generally useful in treatment of an of HCV infection.

Whether a subject method is effective in treating an HCV infection can be determined by a reduction in viral load, a reduction in time to seroconversion (virus undetectable in patient serum), an increase in the rate of sustained viral response to therapy, a reduction of morbidity or mortality in clinical outcomes, or other indicator of disease response.

In general, an effective amount of a compound of Formulas I-IV, and optionally one or more additional antiviral agents, is an amount that is effective to reduce viral load or achieve a sustained viral response to therapy.

Whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

The method involves administering an effective amount of a compound of Formulas I-IV, optionally in combination with an effective amount of one or more additional antiviral agents. In some embodiments, an effective amount of a compound of Formulas I-IV, and optionally one or more additional antiviral agents, is an amount that is effective to reduce viral titers to undetectable levels, e.g., to about 1000 to about 5000, to about 500 to about 1000, or to about 100 to about 500 genome copies/mL serum. In some embodiments, an effective amount of a compound of Formulas I-IV, and optionally one or more additional antiviral agents, is an amount that is effective to reduce viral load to lower than 100 genome copies/mL serum.

In some embodiments, an effective amount of a compound of Formulas I-IV, and optionally one or more additional antiviral agents, is an amount that is effective to achieve a 1.5-log, a 2-log, a 2.5-log, a 3-log, a 3.5-log, a 4-log, a 4.5-log, or a 5-log reduction in viral titer in the serum of the individual.

In many embodiments, an effective amount of a compound of Formulas I-IV, and optionally one or more additional antiviral agents, is an amount that is effective to achieve a sustained viral response, e.g., non-detectable or substantially non-detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

As noted above, whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of a compound of Formulas I-IV, and optionally one or more additional antiviral agents, is an amount effective to reduce ALT levels to less than about 45 IU/ml serum.

A therapeutically effective amount of a compound of Formulas I-IV, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., enzyme-linked immunosorbent assays (ELISA), radioimmunoassays, and the like, using antibody specific for a given serum marker.

In many embodiments, an effective amount of a compound of Formulas I-IV and an additional antiviral agent is a synergistic amount. As used herein, a “synergistic combination” or a “synergistic amount” of a compound of Formulas I-IV and an additional antiviral agent is a combined dosage that is more effective in the therapeutic or prophylactic treatment of an HCV infection than the incremental improvement in treatment outcome that could be predicted or expected from a merely additive combination of (i) the therapeutic or prophylactic benefit of the compound of Formulas I-IV when administered at that same dosage as a monotherapy and (ii) the therapeutic or prophylactic benefit of the additional antiviral agent when administered at the same dosage as a monotherapy.

In some embodiments, a selected amount of a compound of Formulas I-IV and a selected amount of an additional antiviral agent are effective when used in combination therapy for a disease, but the selected amount of the compound of Formulas I-IV and/or the selected amount of the additional antiviral agent is ineffective when used in monotherapy for the disease. Thus, the embodiments encompass (1) regimens in which a selected amount of the additional antiviral agent enhances the therapeutic benefit of a selected amount of the compound of Formulas I-IV when used in combination therapy for a disease, where the selected amount of the additional antiviral agent provides no therapeutic benefit when used in monotherapy for the disease (2) regimens in which a selected amount of the compound of Formulas I-IV enhances the therapeutic benefit of a selected amount of the additional antiviral agent when used in combination therapy for a disease, where the selected amount of the compound of Formulas I-IV provides no therapeutic benefit when used in monotherapy for the disease and (3) regimens in which a selected amount of the compound of Formulas I-IV and a selected amount of the additional antiviral agent provide a therapeutic benefit when used in combination therapy for a disease, where each of the selected amounts of the compound of Formulas I-IV and the additional antiviral agent, respectively, provides no therapeutic benefit when used in monotherapy for the disease. As used herein, a “synergistically effective amount” of a compound of Formulas I-IV and an additional antiviral agent, and its grammatical equivalents, shall be understood to include any regimen encompassed by any of (1)-(3) above.

### Fibrosis

The embodiments provides methods for treating liver fibrosis (including forms of liver fibrosis resulting from, or associated with, HCV infection), generally involving administering a therapeutic amount of a compound of Formulas I-IV, and optionally one or more additional antiviral agents. Effective amounts of compounds of Formulas I-IV, with and without one or more additional antiviral agents, as well as dosing regimens, are as discussed below.

Whether treatment with a compound of Formulas I-IV, and optionally one or more additional antiviral agents, is effective in reducing liver fibrosis is determined by any of a number of well-established techniques for measuring liver fibrosis and liver function. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of a compound of Formulas I-IV, and optionally one or more additional antiviral agents, is an amount that effects a change of one unit or more in the fibrosis stage based on pre- and post-therapy liver biopsies. In particular embodiments, a therapeutically effective amount of a compound of Formulas I-IV, and optionally one or more additional antiviral agents, reduces liver fibrosis by at least one unit in the METAVIR, the Knodell, the Scheuer, the Ludwig, or the Ishak scoring system.

Secondary, or indirect, indices of liver function can also be used to evaluate the efficacy of treatment with a compound of Formulas I-IV. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

An effective amount of a compound of Formulas I-IV, and optionally one or more additional antiviral agents, is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of a compound of Formulas I-IV, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Quantitative tests of functional liver reserve can also be used to assess the efficacy of treatment with an interferon receptor agonist and pirfenidone (or a pirfenidone analog). These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of a compound of Formulas I-IV, and optionally one or more additional antiviral agents, is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with a compound of Formulas I-IV, and optionally one or more additional antiviral agents, is effective in reducing the incidence of a disorder associated with cirrhosis of the liver can readily be determined by those skilled in the art.

Reduction in liver fibrosis increases liver function. Thus, the embodiments provide methods for increasing liver function, generally involving administering a therapeutically effective amount of a compound of Formulas I-IV, and optionally one or more additional antiviral agents. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal level of alanine transaminase is about 45 IU per milliliter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of a compound of Formulas I-IV, and optionally one or more additional antiviral agents, is one that is effective to increase liver function by at least about 10%, at least about 20%, at least about 30%, at least about 40%, at least about 50%, at least about 60%, at least about 70%, at least about 80%, or more. For example, a therapeutically effective amount of a compound of Formulas I-IV, and optionally one or more additional antiviral agents, is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of a compound of Formulas I-IV, and optionally one or more additional antiviral agents, is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

### Dosages, Formulations, and Routes of Administration

In the subject methods, the active agent(s) (e.g., compound of Formulas I-IV, and optionally one or more additional antiviral agents) may be administered to the host using any convenient means capable of resulting in the desired therapeutic effect. Thus, the agent can be incorporated into a variety of formulations for therapeutic administration. More particularly, the agents of the embodiments can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

### Formulations

The above-discussed active agent(s) can be formulated using well-known reagents and methods. Compositions are provided in formulation with a pharmaceutically acceptable excipient(s). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, an agent is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

As such, administration of the agents can be achieved in various ways, including oral, buccal, rectal, parenteral, intraperitoneal, intradermal, subcutaneous, intramuscular, transdermal, intratracheal, etc., administration. In many embodiments, administration is by bolus injection, e.g., subcutaneous bolus injection, intramuscular bolus injection, and the like.

The pharmaceutical compositions of the embodiments can be administered orally, parenterally or via an implanted reservoir. Oral administration or administration by injection is preferred.

Subcutaneous administration of a pharmaceutical composition of the embodiments is accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a pharmaceutical composition of the embodiments to a patient through the port is referred to herein as “a subcutaneous injection port delivery system.” In many embodiments, subcutaneous administration is achieved by bolus delivery by needle and syringe.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. The compounds of the embodiments can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the embodiments calculated in an amount sufficient to produce the desired effect in association with a pharmaceutically acceptable diluent, carrier or vehicle. The specifications for the novel unit dosage forms of the embodiments depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Coadministration with Other Antiviral or Antifibrotic Agents

A subject method will in some embodiments be carried out by administering an NS3 inhibitor that is a compound of Formulas I-IV, and optionally one or more additional antiviral agent(s).

In some embodiments, the method further includes administration of one or more interferon receptor agonist(s). In other embodiments, the method further includes administration of pirfenidone or a pirfenidone analog.

Additional antiviral agents that are suitable for use in combination therapy include, but are not limited to, nucleotide and nucleoside analogs. Non-limiting examples include azidothymidine (AZT) (zidovudine), and analogs and derivatives thereof; 2′,3′-dideoxyinosine (DDI) (didanosine), and analogs and derivatives thereof; 2′,3′-dideoxycytidine (DDC) (dideoxycytidine), and analogs and derivatives thereof; 2′,3′-didehydro-2′,3′-dideoxythymidine (D4T) (stavudine), and analogs and derivatives thereof; combivir; abacavir; adefovir dipoxil; cidofovir; ribavirin; ribavirin analogs; and the like.

In some embodiments, the method further includes administration of ribavirin. Ribavirin, 1-β-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. Some embodiments also involve use of derivatives of ribavirin (see, e.g., U.S. Pat. No. 6,277,830). The ribavirin may be administered orally in capsule or tablet form, or in the same or different administration form and in the same or different route as the interferon receptor agonist. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, an additional antiviral agent is administered during the entire course of NS3 inhibitor compound treatment. In other embodiments, an additional antiviral agent is administered for a period of time that is overlapping with that of the NS3 inhibitor compound treatment, e.g., the additional antiviral agent treatment can begin before the NS3 inhibitor compound treatment begins and end before the NS3 inhibitor compound treatment ends; the additional antiviral agent treatment can begin after the NS3 inhibitor compound treatment begins and end after the NS3 inhibitor compound treatment ends; the additional antiviral agent treatment can begin after the NS3 inhibitor compound treatment begins and end before the NS3 inhibitor compound treatment ends; or the additional antiviral agent treatment can begin before the NS3 inhibitor compound treatment begins and end after the NS3 inhibitor compound treatment ends.

### Methods of Treatment

### Monotherapies

The NS3 inhibitor compounds described herein may be used in acute or chronic therapy for HCV disease. In many embodiments, the NS3 inhibitor compound is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The NS3 inhibitor compound can be administered 5 times per day, 4 times per day, tid, bid, qd, qod, biw, tiw, qw, qow, three times per month, or once monthly. In other embodiments, the NS3 inhibitor compound is administered as a continuous infusion.

In many embodiments, an NS3 inhibitor compound of the embodiments is administered orally.

In connection with the above-described methods for the treatment of HCV disease in a patient, an NS3 inhibitor compound as described herein may be administered to the patient at a dosage from about 0.01 mg to about 100 mg/kg patient bodyweight per day, in 1 to 5 divided doses per day. In some embodiments, the NS3 inhibitor compound is administered at a dosage of about 0.5 mg to about 75 mg/kg patient bodyweight per day, in 1 to 5 divided doses per day.

The amount of active ingredient that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific NS3 inhibitor compound, the severity of the symptoms and the susceptibility of the subject to side effects. Preferred dosages for a given NS3 inhibitor compound are readily determinable by those of skill in the art by a variety of means. A preferred means is to measure the physiological potency of a given interferon receptor agonist.

In many embodiments, multiple doses of NS3 inhibitor compound are administered. For example, an NS3 inhibitor compound is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### Patient Identification

In certain embodiments, the specific regimen of drug therapy used in treatment of the HCV patient is selected according to certain disease parameters exhibited by the patient, such as the initial viral load, genotype of the HCV infection in the patient, liver histology and/or stage of liver fibrosis in the patient.

Thus, some embodiments provide any of the above-described methods for the treatment of HCV infection in which the subject method is modified to treat a treatment failure patient for a duration of 48 weeks.

Other embodiments provide any of the above-described methods for HCV in which the subject method is modified to treat a non-responder patient, where the patient receives a 48 week course of therapy.

Other embodiments provide any of the above-described methods for the treatment of HCV infection in which the subject method is modified to treat a relapser patient, where the patient receives a 48 week course of therapy.

Other embodiments provide any of the above-described methods for the treatment of HCV infection in which the subject method is modified to treat a naïve patient infected with HCV genotype 1, where the patient receives a 48 week course of therapy.

Other embodiments provide any of the above-described methods for the treatment of HCV infection in which the subject method is modified to treat a naïve patient infected with HCV genotype 4, where the patient receives a 48 week course of therapy.

Other embodiments provide any of the above-described methods for the treatment of HCV infection in which the subject method is modified to treat a naïve patient infected with HCV genotype 1, where the patient has a high viral load (HVL), where “HVL” refers to an HCV viral load of greater than 2×10⁶ HCV genome copies per mL serum, and where the patient receives a 48 week course of therapy.

One embodiment provides any of the above-described methods for the treatment of an HCV infection, where the subject method is modified to include the steps of (1) identifying a patient having advanced or severe stage liver fibrosis as measured by a Knodell score of 3 or 4 and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having advanced or severe stage liver fibrosis as measured by a Knodell score of 3 or 4 and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per ml of patient serum and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per ml of patient serum and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per ml of patient serum and no or early stage liver fibrosis as measured by a Knodell score of 0, 1, or 2 and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per ml of patient serum and no or early stage liver fibrosis as measured by a Knodell score of 0, 1, or 2 and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 20 weeks to about 24 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 24 weeks to about 48 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 20 weeks to about 24 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of at least about 24 weeks.

Another embodiment provides any of the above-described methods for the treatment of an HCV infection, where the subject method is modified to include the steps of (1) identifying a patient having an HCV genotype 1 or 4 infection and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV infection characterized by any of HCV genotypes 5, 6, 7, 8 and 9 and then (2) administering to the patient the drug therapy of the subject method for a time period of about 20 weeks to about 50 weeks.

Another embodiment provides any of the above-described methods for the treatment of an HCV infection, where the subject method is modified to include the steps of (1) identifying a patient having an HCV infection characterized by any of HCV genotypes 5, 6, 7, 8 and 9 and then (2) administering to the patient the drug therapy of the subject method for a time period of at least about 24 weeks and up to about 48 weeks.

### Subjects Suitable for Treatment

Any of the above treatment regimens can be administered to individuals who have been diagnosed with an HCV infection. Any of the above treatment regimens can be administered to individuals who have failed previous treatment for HCV infection (“treatment failure patients,” including non-responders and relapsers).

Individuals who have been clinically diagnosed as infected with HCV are of particular interest in many embodiments. Individuals who are infected with HCV are identified as having HCV RNA in their blood, and/or having anti-HCV antibody in their serum. Such individuals include anti-HCV ELISA-positive individuals, and individuals with a positive recombinant immunoblot assay (RIBA). Such individuals may also, but need not, have elevated serum ALT levels.

Individuals who are clinically diagnosed as infected with HCV include naïve individuals (e.g., individuals not previously treated for HCV, particularly those who have not previously received IFN-α-based and/or ribavirin-based therapy) and individuals who have failed prior treatment for HCV (“treatment failure” patients). Treatment failure patients include non-responders (i.e., individuals in whom the HCV titer was not significantly or sufficiently reduced by a previous treatment for HCV, e.g., a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-α monotherapy, a previous IFN-α and ribavirin combination therapy, or a previous pegylated IFN-α and ribavirin combination therapy, whose HCV titer decreased, and subsequently increased).

In particular embodiments of interest, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including Ia and Ib, 2, 3, 4, 6, etc. and subtypes (e.g., 2a, 2b, 3a, etc.)), particularly a difficult to treat genotype such as HCV genotype 1 and particular HCV subtypes and quasispecies.

Also of interest are HCV-positive individuals (as described above) who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In particular embodiments of interest, HCV-positive individuals with stage 3 or 4 liver fibrosis according to the METAVIR scoring system are suitable for treatment with the methods described herein. In other embodiments, individuals suitable for treatment with the methods of the embodiments are patients with decompensated cirrhosis with clinical manifestations, including patients with far-advanced liver cirrhosis, including those awaiting liver transplantation. In still other embodiments, individuals suitable for treatment with the methods described herein include patients with milder degrees of fibrosis including those with early fibrosis (stages 1 and 2 in the METAVIR, Ludwig, and Scheuer scoring systems; or stages 1, 2, or 3 in the Ishak scoring system.).

### Preparation of NS3 Inhibitors

The NS3 inhibitors in the following sections can be prepared according to the procedures and schemes shown in each section. The numberings in each Preparation of NS3 Inhibitor Section are meant for that specific section only, and should not be construed as or confused with same numberings in other sections.

### Methodology

### Preparation of NS3 Inhibitors

The HCV helicase inhibitors can be prepared according to the procedures and schemes shown below.

The synthesis of the NS3 helicase inhibitors having Formula I is summarized in Scheme 1. The general procedure below describes the reaction conditions for the synthesis of these compounds. R₃ may be an alkyl group, for example a methyl or an ethyl group.

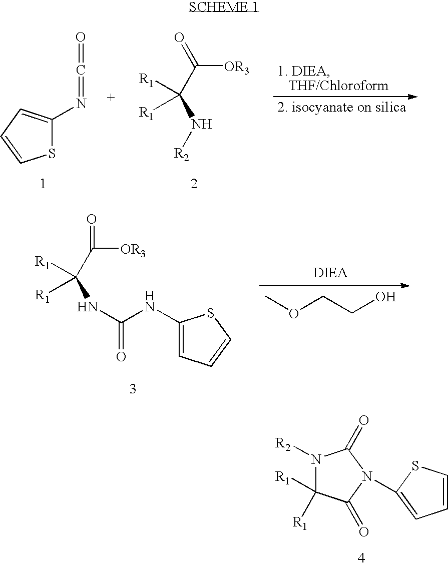

### General Procedure for the Synthesis of Compounds of Formula I

A solution of 2-isocyanatothiophene 1 (0.125 mmol/L in THF) is added to a solution of the amino ester 2 (1.2 equiv) and the DIEA (1 ml/mmol of 2) in an appropriate amount of chloroform. The reaction is shaken at room temperature until all the isocyanate has been consumed (typically 2-24 h). Isocyanate on silica (5 equiv.) is added and the reaction is shaken at room temperature until the excess amino ester has been trapped (typically 6-24 h). The reaction is filtered and the filtrate concentrated in vacuo. The obtained residue is taken up in an appropriate amount of 2-methoxyethanol and DIEA (1 ml/mmol of 1) added. The reaction is shaken at room temperature for 24-36 h. At this point the reaction mixture is checked by LC-MS for remaining uncyclized product 3. If significant amounts of 3 are found, the reaction mixture is heated to 60° C. until cyclization is complete. Once no intermediate 3 is detected, the reaction is concentrated in vacuo to obtain the crude product. If the crude product is not sufficiently pure, it can be purified using normal or reverse phase chromatography.

The NS3 helicase inhibitors having Formula I shown in Table 3 were prepared as described above.

TABLE 3

| | | LCMS |
|---|---|---|
| Compound | Structure | (m/z) |
| 5 | 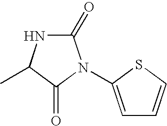 | 195.0 |
| 6 | 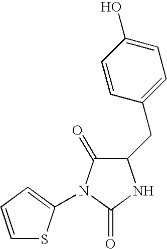 | 289.0 |
| 7 | 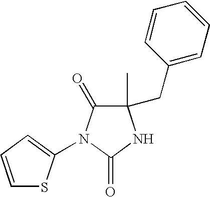 | 287.0 |
| 8 | 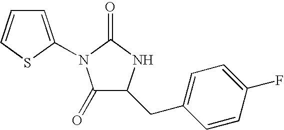 | 291.0 |
| 9 | 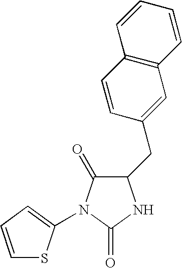 | 323.0 |
| 10 | 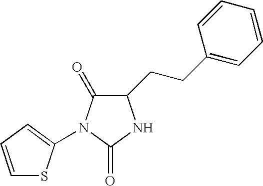 | 287.0 |
| 11 | 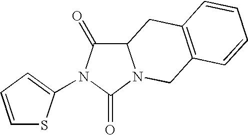 | 285.2 |
| 12 | 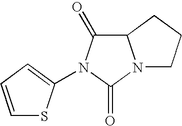 | 223.0 |
| 13 | 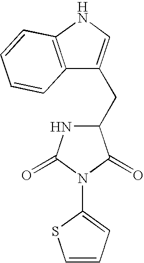 | 312.0 |
| 14 | 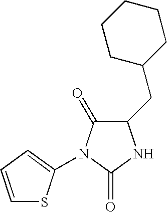 | 279.0 |
| 15 | 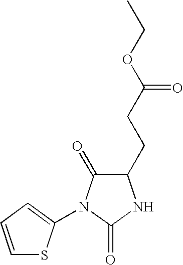 | 282.9 |
| 16 | 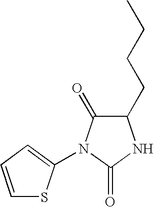 | 239.0 |
| 17 | 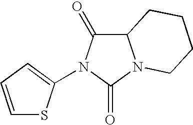 | 237.1 |
| 18 | 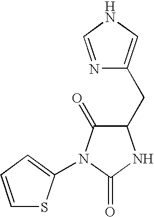 | 263.1 |
| 19 | 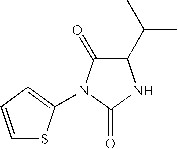 | 225.0 |
| 20 | 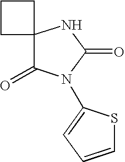 | 223.0 |

### General Procedure for the Synthesis of Compounds of Formula II

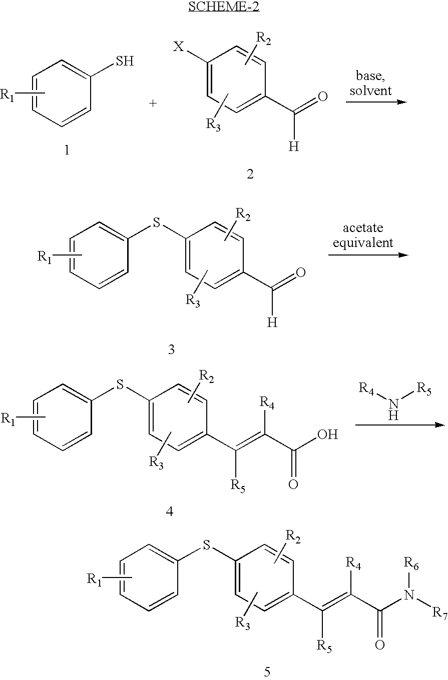

Substituted aryl cinnamide analogue 5 may be prepared according to the method illustrated in Scheme-1 using published protocols in the literature (WO/00139081, Marty Winn et al., J. Med. Chem. 2001, 44(25), 4393-4403)

Diarylsulfide intermediate 3 may be prepared by the reaction of various substituted halo benzaldehydes (such 2 or 4-fluorobenzaldehyde, 2 or 4-chlorobenzaldehyde) with various substitute thiophenols (eg: 4-fluorothiophenol, 2-methoxy thiophenols or the like) in the presence of a suitable base such as potassium carbonate, sodium carbonate, triethylamine or the like in a polar solvent (for example DMF, DMA, acetone, methanol and the like). The resulting diarylsulfide aldehyde 3 can be reacted with an acetate equivalent such as malonic acid or triethoxyphosphonoacetate or other similar reagents to provide the cinnamic acid 4 or the corresponding ester. In the case of the ester, it may be hydrolyzed with an inorganic base (such as LiOH, NaOH, KOH or the like) in a mixture of an alcohol (for example ethanol, methanol) and water to provide the acid 4. The coupling of the cinnamic acid 4 with a primary or secondary amine under standard amide bond formation conditions (which includes the activation of the acid using thionyl chloride, or dicyclohexylcarbodiimide and N-hydroxysuccinimide, or the like) can provide the final cinnamide analogue 5.

Alternatively the compound 8 may be prepared from the sequence shown in Scheme 3.

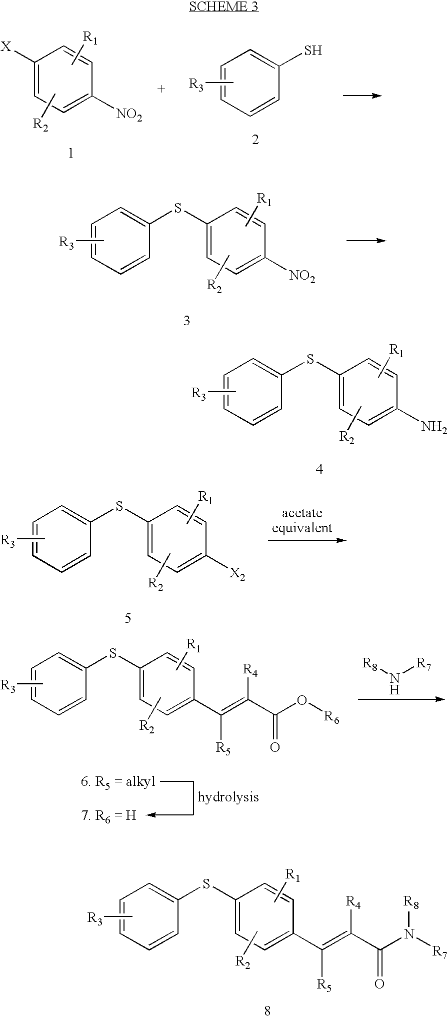

A substituted para-nitro halo benzene analogue 1 may be reacted with a substituted arylthiol 2 (such as 4-fluorobenzenethiol, 2-methoxybenzene thiol and the like) in the presence of a suitable base such as potassium carbonate, sodium carbonate, triethylamine or the like in a polar solvent (for example DMF, DMA, acetone, methanol and the like) to provide the intermediate 3. The intermediate 3 may be converted to the to the corresponding aniline 4 by hydrogenation employing a catalyst such as Pd/C, Pt/C, Pd(OH)₂, Pd(OAc)₂ and the like or with the use of Zn/EtOH, SnCl₂, or the alike. Aniline 5 may be converted to the corresponding iodo or bromo analogue 5 by standard Sandmeyer reaction conditions published in the literature. The cinnamide analogue 6 may be prepared from the reaction of 5 with an acetate equivalent such triethoxyphosphonoacetate or other similar reagents. The resulting ester 6 may hydrolyzed to the corresponding acid 7 using an inorganic base (such as LiOH, NaOH, KOH or the like) in a mixture of an alcohol (for example ethanol, methanol) and water. The final diarylsulfide compound 8 may be prepared from the reaction of acid 7 with a primary or secondary amine under standard amide bond formation conditions (which includes the activation of the acid using thionyl chloride, or dicyclohexylcarbodiimide and N-hydroxysuccinimide, or the like).

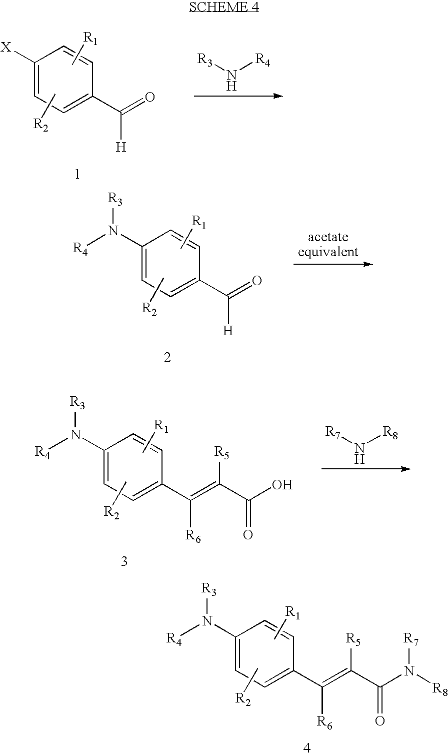

Scheme 3 illustrates the preparation of amino substituted cinnamides 4. The halosubstituted benzaldehyde 1 (such as such 2 or 4-fluoro benzaldehyde, 2 or 4-chlorobenzaldehyde) may be reacted with a primary or secondary amine (for example methylamine, dimethylamine, morpholine, piperidine, substituted piperazines and the like) in the presence of a suitable base (such as potassium carbonate, sodium carbonate, triethylamine or the like) in a polar solvent (for example DMF, DMA, acetone, methanol and the like). The resulting aldehyde 2 may be reacted with an acetate equivalent such as malonic acid or triethoxyphosphonoacetate or other similar reagents to provide the cinnamic acid 3 or the corresponding ester. In the case of the ester, it can be hydrolyzed with an inorganic base (such as LiOH, NaOH, KOH or the like) in a mixture of an alcohol (for example ethanol, methanol) and water to provide the acid 3. The coupling of the cinnamic acid 3 with a primary or secondary amine under standard amide bond formation conditions (which includes the activation of the acid using thionyl chloride, or dicyclohexylcarbodiimide and N-hydroxysuccinimide, or the like) can provide the final cinnamide analogue 4.

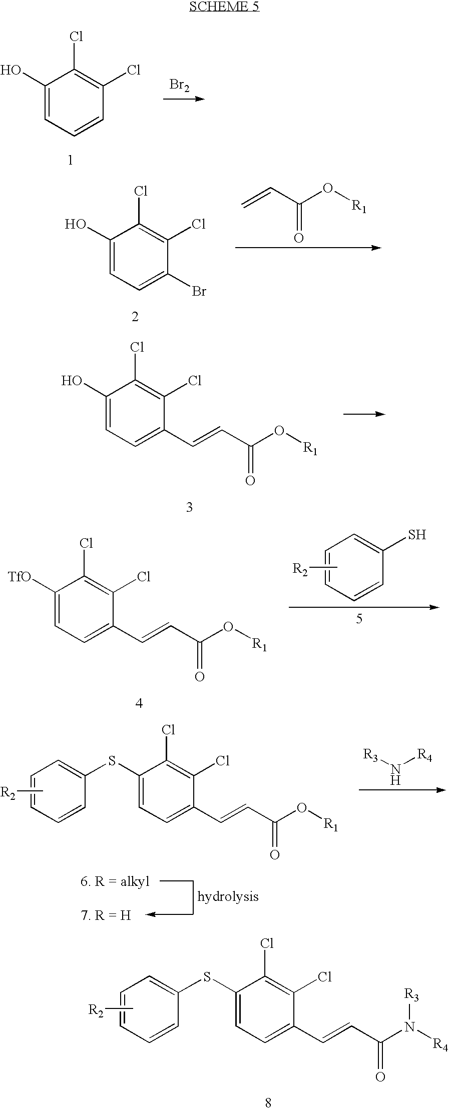

2,3-Dichloro substituted diarylsulfide 8 may be prepared from the reaction sequence described in the literature (WO/00139081). The bromide 2 may be prepared from the bromination of phenol 1 with Br₂ in a non polar solvent like CH₂Cl₂ or CHCl₃ at a lower temperature (0° C. to room temperature). Then Heck coupling of this intermediate with alkyl acrylate would furnish the intermediate 3. The phenol 3 may be converted to the triflate 4 using triflicanhydride in CH₂Cl₂ or CHCl₃ at a lower temperature (0° C. to −20° C.) in the presence of a base such as Hunig's base, triethylamine, lutidine or the like. The coupling of the triflate 4 with a thiophenol 5 can be carried out in the presence of a base (LiO^{t}Bu, KO^{t}Bu or the like) in a polar solvent (DMF, NMP or the like) to provide the diarylsulfide analogue 6. Hydrolysis of the ester 6 may be achieved using a base such as LiOH, NaOH, KOH or the like in a mixture of solvents (for example EtOH/water, MeOH/water, THF:MeOH/water or a similar solvent system). The coupling of the cinnamic acid 7 with a primary or secondary amine under standard amide bond formation conditions (which includes the activation of the acid using thionyl chloride, or dicyclohexylcarbodiimide and N-hydroxysuccinimide, or the like) may provide the final cinnamide analogue 8.

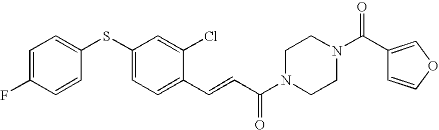

### EXAMPLE 1

### (E)-3-(2-chloro-4-(4-fluorophenylthio)phenyl)-1-(4-(furan-3-carbonyl)piperazin-1-yl)prop-2-en-1-one

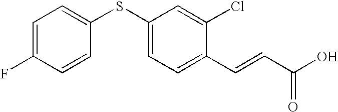

### EXAMPLE 1A

### (E)-3-(2-chloro-4-(4-fluorophenylthio)-phenyl)acrylic acid

Example 1A was prepared from the reaction of 4-fluorobenzenethiol with 2-chloro-4-fluorobenzaldehyde followed by condensation with malonic acid according to the procedure described by Marty Winn et al., J. Med. Chem. 2001, 44(25), 4393-4403.

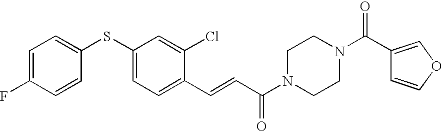

### EXAMPLE 1B

### (E)-3-(2-chloro-4-(4-fluorophenylthio)phenyl)-1-(4-(furan-3-carbonyl)piperazin-1-yl)prop-2-en-1-one

A solution of Example 1A (60 mg, 0.194 mmol), HOBt.H₂O (44.64 mg, 0.2915 mmol), N-methylmorpholine (64 μM, 0.583 mmol), and furan-3-yl(piperazin-1-yl)methanone (42 mg, 0.233 mmol) in DMF (1 mL) was treated EDCI (56 mg, 0.292 mmol) and stirred at ambient temperature. After 18 h the mixture was diluted with CH₂Cl₂ (2 mL) and washed with water (2 mL). The CH₂Cl₂ layer was separated and directly purified by flash chromatography on silica gel (5 g Alltech SEP packs) eluting with a step gradient of 30% EtOAc/hexane to provide the title compound (38 mg, 42% yield) as a white solid. LCMS (APCI)⁻ at m/z 469 (M−H)⁻.

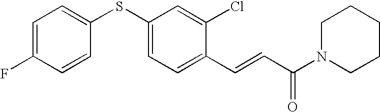

### EXAMPLE 2

### (E)-3-(2-chloro-4-(4-fluorophenylthio)phenyl)-1-(piperidin-1-yl)prop-2-en-1-one

Example 2 was prepared as described for Example 1A (60 mg, 0.194 mmol), except substituting and furan-3-yl(piperazin-1-yl)methanone with piperidine. The product was isolated in 68% yield (49 mg) after silica gel flash chromatography. LCMS (APCI)⁻ at m/z 374 (M−H)⁻, Rₜ=4.32 min.

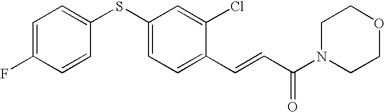

### EXAMPLE 3

### (E)-3-(2-chloro-4-(4-fluorophenylthio)phenyl)-1-morpholinoprop-2-en-1-one

Example 3 was prepared from Example 1A (60 mg, 0.194 mmol), according to the method described for Example 1B, except substituting and furan-3-yl(piperazin-1-yl)methanone with morpholine. LCMS (APCI)⁻ at m/z 378 (M−H)⁻, Rₜ=3.82 min.

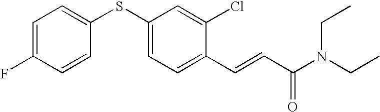

### EXAMPLE 4

### (E)-3-(2-chloro-4-(4-fluorophenylthio)phenyl)-N,N-diethylacrylamide

Example 4 (37 mg) was prepared from Example 1A (60 mg, 0.194 mmol), according to the method described for Example 1B, except substituting and furan-3-yl(piperazin-1-yl)methanone with diethylamine. LCMS (APCI)⁻ at m/z 362 (M−H)⁻, Rₜ=4.25 mm.

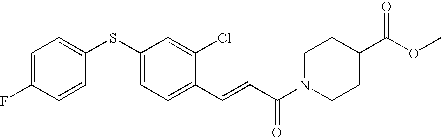

### EXAMPLE 5

### (E)-methyl 1-(3-(2-chloro-4-(4-fluorophenylthio)phenyl)acryloyl)piperidine-4-carboxylate

Example 5 (46 mg) was prepared from Example 1A (60 mg, 0.194 mmol), according to the method described for Example 1B, except substituting and furan-3-yl(piperazin-1-yl)methanone with methyl isonipecotate. LCMS (APCI)⁻ at m/z 432 (M−H)⁻, Rₜ=4.08 min.

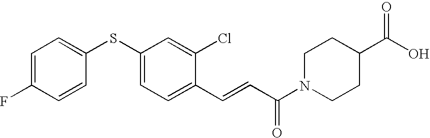

### EXAMPLE 6

### (E)-1-(3-(2-chloro-4-(4-fluorophenylthio)phenyl)acryloyl-4-carboxylic acid

Hydrolysis of Example 5 (40 mg, 0.095 mmol) with LiOH.H₂O was carried out according to the procedure described in J. Med. Chem. 2001, 44(25), 4393-4403 by Marty Winn at el to provide the title compound as a white powder. LCMS (APCI)⁻ at m/z 426 (M−H)⁻, Rₜ=2.99 min.

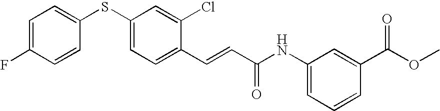

### EXAMPLE 7

### (E)-methyl 3-(3-(2-chloro-4-(4-fluorophenylthio)phenyl)acrylamido)benzoate

The title compound was prepared from Example 1A as described in Example 1B except substituting furan-3-yl(piperazin-1-yl)methanone with methyl 3-amino benzoate. Example 7 was isolated as a white powder. LCMS (APCI)⁻ at m/z 441 (M−H)⁻.

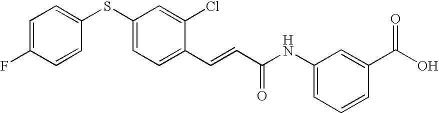

### EXAMPLE 8

### (E)-3-(3-(2-chloro-4-(4-fluorophenylthio)phenyl)acrylamido)benzoic acid

Example 8 was prepared from Example 7 according the method described for Example 6. Title compound was obtained as a white powder. LCMS (APCI)⁻ at m/z 418 (M−H)⁻, Rₜ=2.79 min.

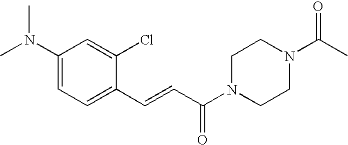

### EXAMPLE 9

### (E)-1-(4-acetylpiperazin-1-yl)-3-(2-chloro-4-(dimethylamino)phenyl)prop-2-en-1-one

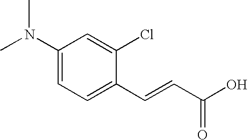

### EXAMPLE 9A

### (E)-3-(2-chloro-4-(dimethylamino)phenyl)acrylic acid

Example 9A was prepared from the reaction of 2-chloro-4-fluorobenzaldehyde with dimethylamine followed by subsequent condensation with malonic acid as described for Example 1A.

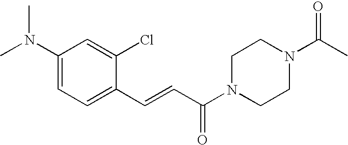

### EXAMPLE 9B

### (E)-1-(4-acetylpiperazin-1-yl)-3-(2-chloro-4-(dimethylamino)phenyl)prop-2-en-1-one

Title compound (115 mg) was prepared from Example 9A as described for Example 1B except substituting furan-3-yl(piperazin-1-yl)methanone with 1-(piperazin-1-yl)ethanone. LCMS (APCI)⁺ at m/z 336 (M+H)⁺, Rt=2.73 min.

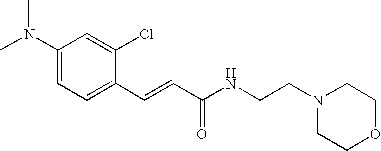

### EXAMPLE 10

### (E)-3-(2-chloro-4-(dimethylaminophenyl-N-(2-morpholinoethyl)acrylamide

Example 9A was processed as in Example 1B except substituting furan-3-yl(piperazin-1-yl)methanone with 2-morpholinoethanamine to provide the title compound (27.4 mg). LCMS (APCI)⁺ at m/z 338 (M+H)⁺, Rt=2.64 min.

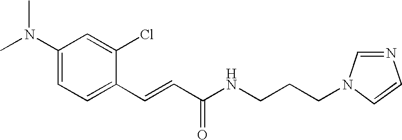

### EXAMPLE 11

### (E)-N-(3-(1H-imidazol-1-yl)propyl)-3-(2-chloro-4-(dimethylamino)phenyl)acrylamide

Example 11 (15 mg) was prepared from Example 9A as described for the Example 1B except substituting furan-3-yl(piperazin-1-yl)methanone with 3-(1H-imidazol-1-yl)propan-1-amine. LCMS (APCI)⁺ at m/z 333 (M+H)⁺, Rt=2.62 min.

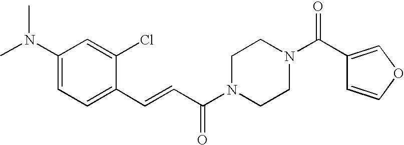

### EXAMPLE 12

### (E)-3-(2-chloro-4-(dimethylamino)phenyl)-1-(4-(furan-3-carbonyl)piperazin-1-yl)prop-2-en-1-one

Example 12 (69 mg) was prepared from Example 9A according to the method described for Example 1B. LCMS (APCI)⁺ at m/z 388 (M+H)⁺, Rt=3.81 min.

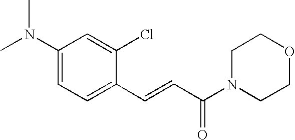

### EXAMPLE 13

### (E)-3-(2-chloro-4-(dimethylamino)phenyl)-1-morpholinoprop-2-en-1-one

Example 13 (51.9 mg) was prepared according to the method described for Example 1B except substitution furan-3-yl(piperazin-1-yl)methanone with morpholine. LCMS (APCI)⁺ at m/z 295 (M+H)⁺, Rt=3.01 min.

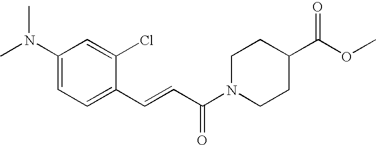

### EXAMPLE 14

### (E)-methyl 1-(3-(2-chloro-4-(dimethylamino)phenyl)acryloyl)piperidine-4-carboxylate

Title compound (73 mg) was prepared from Example 9A as described for Example 5. LCMS (APCI)⁺ at m/z 351 (M+H)⁺, Rt=3.32 min.

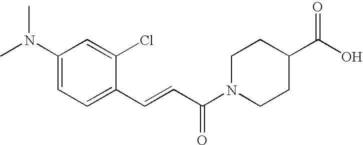

### EXAMPLE 15

### (E)-1-(3-(2-chloro-4-(dimethylaminophenyl)acryloyl-4-carboxylic acid

Example 15 (24 mg) was prepared from Example 14 as described for Example 6. LCMS (APCI)⁺ at m/z 335 (M−H)⁻, Rt=2.19 min.

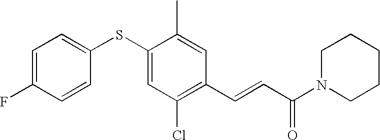

### EXAMPLE 16

### (E)-3-(2-chloro-4-(4-fluorophenylthio)-5-methylphenyl)-1-(piperidin-1-yl)prop-2-en-1-one

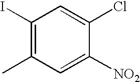

### EXAMPLE 16A

### 1-chloro-5-iodo-4-methyl-2-nitrobenzene

Example 16A was prepared from 5-chloro-2-methyl-4-nitroaniline according to the method described in Tetrahedron Letters, 2005, 46 (18), 3197. ¹H NMR (400 MHz, DMSO d₆) δ 8.267 (s, 1H), 8.062 (s, 1H), 2.434 (s, 3H).

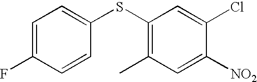

### EXAMPLE 16B

### (5-chloro-2-methyl-4-nitrophenyl)(4-fluorophenyl)sulfane

Example 16A was treated with 4-fluorobenzenethiol as described in Organic Letters, 2002, 9(20), 3517 to provided the title compound in 87% yield. ¹H NMR (400 MHz, DMSO d₆) δ 8.073 (s, 1H), 7.687-7.647 (m, 2H), 7.45-7.40 (m, 2H), 6.79 (s, 1H), 2.38 (s, 3H).

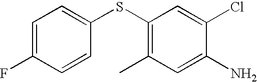

### EXAMPLE 16C

### 2-chloro-4-(4-fluorophenylthio)-5-methylaniline

Example 16B was reduced according to the method described in Bioorganic & Medicinal Chemistry Letters, 2005, 15(8), 2033-2039 to provide the title compound as a colorless oil (99% yield). ¹H NMR (400 MHz, DMSO d₆) δ 7.32 (s, 1H), 7.15-7.11 (m, 2H), 7.05-7.02 (m, 2H), 6.77 (s, 1H), 5.71 (br s, 2H), 2.15 (s, 3H).

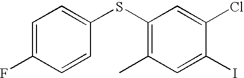

### EXAMPLE 16D

### (5-chloro-4-iodo-2-methylphenyl)(4-fluorophenyl)sulfane

Example 16D was prepared from Example 16C (1.8 g, 6.723 mmol) according to the method described for Example 16A. ¹H NMR (400 MHz, DMSO d₆) δ 7.85 (s, 1H), 7.4-7.41 (m, 2H), 7.29-7.24 (m, 2H), 6.99 (s, 1H), 2.22 (s, 3H).

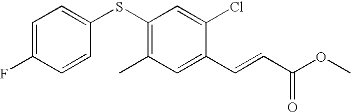

### EXAMPLE 16E

### (E)-methyl 3-(2-chloro-4-(4-fluorophenylthio)-5-methylphenyl)acrylate

Example 16E was prepared from the reaction of Example 16D with methyl acrylate according to a procedure described in WO/00139081. ¹H NMR (400 MHz, DMSO d₆) δ 7.89 (s, 1H), 7.77 (d, J=16.01 Hz, 1H), 7.54-7.51 (m, 2H), 7.35-7.31 (m, 2H), 6.79 (s, 1H), 6.69 (d, J=16.01 Hz, 1H), 3.71 (s, 3H), 2.29 (s, 3H).

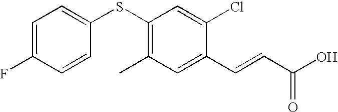

### EXAMPLE 16F

### (E)-3-(2-chloro-4-(4-fluorophenylthio)-5-methylphenyl)acrylic acid

Example 16E (400 mg, 1.188 mmol) was treated with LiOH.H₂O as described for Example 6 to provide the title compound in 93% yield (300 mg). ¹H NMR (400 MHz, DMSO d₆) δ 12.61 (s, 1H), 7.89 (s, 1H), 7.75 (d, J=16.01 Hz, 1H), 7.56-7.53 (m, 2H), 7.37-7.33 (m 2H), 6.83 (s, 1H), 6.61 (d, J=16.01 Hz, 1H), 2.32 (s, 3H).

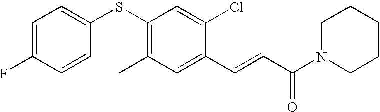

### EXAMPLE 16G

### (E)-3-(2-chloro-4-(4-fluorophenylthio)-5-methylphenyl)-1-(piperidin-1-yl)prop-2-en-1-one

Example 16G (53 mg) was prepared from the Example 16F according to the method described for Example 1B except substituting 3-yl(piperazin-1-yl)methanone with piperidine. LCMS (APCI)⁺ at m/z 390 (M+H)⁺, Rₜ=4.56 min.

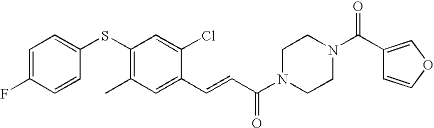

### EXAMPLE 17

### (E)-3-(2-chloro-4-(4-fluorophenylthio)-5-methylphenyl)-1-(4-(furan-3-carbonyl)piperazin-1-yl)prop-2-en-1-one

Title compound (34 mg) was prepared from Example 16F as described for Example 1B. LCMS (APCI)⁺ at m/z 485 (M+H)⁺, Rₜ=4.02 min.

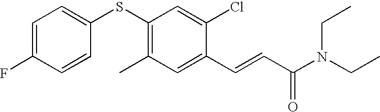

### EXAMPLE 18

### (E)-3-(2-chloro-4-(4-fluorophenylthio)-5-methylphenyl)-N,N-diethylacrylamide

Example 16F was processed as in Example 4 to provide the title compound (49 mg). LCMS (APCI)⁺ at m/z 378 (M+H)⁺, Rₜ=4.44 min.

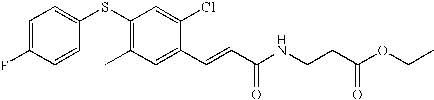

### EXAMPLE 19

### (E)-ethyl 3-(3-(2-chloro-4-(4-fluorophenylthio)-5-methylphenyl)acrylamido)propanoate

Example 19 was prepared from Example 16F according to the method described for Example 1B except substituting furan-3-yl(piperazin-1-yl)methanone with ethyl 3-aminopropanoate. LCMS (APCI)⁺ at m/z 421 (M+H)⁺, Rₜ=4.21 min.

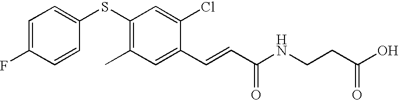

### EXAMPLE 20

### (E)-3-(3-(2-chloro-4-(4-fluorophenylthio)-5-methylphenyl)acrylamido)propanoic acid

Example 20 was processed as in Example 6 to provide the title compound (39 mg). LCMS (APCI)⁻ at m/z 392 [(M−H)⁻, Rₜ=2.74 min.

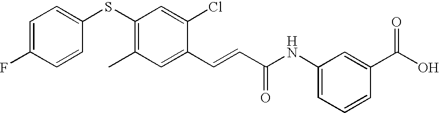

### EXAMPLE 21

### (E)-3-(3-(2-chloro-4-(4-fluorophenylthio)-5-methylphenyl)acrylamido)benzoic acid

Example 21 (12 mg) was prepared from Example 16F and methyl 3-aminobenzoate followed by treatment of LiOH.H₂O according to the method described for the preparation of Example 8. LCMS (APCI)⁻ at m/z 440 (M−H)⁻, Rₜ=3.12 min.

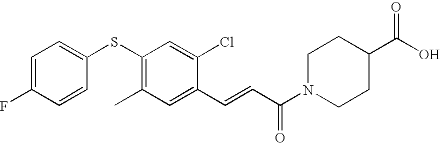

### EXAMPLE 22

### (E)-1-(3-(2-chloro-4-(4-fluorophenylthio)-5-methylphenyl)acryloyl)piperidine-4-carboxylic acid

Example 22 (47 mg) was prepared from Example 16F as described for the Example 6. LCMS (APCI)⁻ at m/z 432 (M−H)⁻, Rₜ=2.88 min.

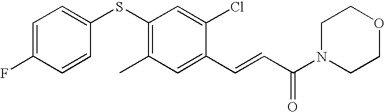

### EXAMPLE 23

### (E)-3-(2-chloro-4-(4-fluorophenylthio)-5-methylphenyl)-1-morpholinoprop-2-en-1-one

Example 23 (52 mg) was prepared from Example 16F according to the procedure describe for Example 3. LCMS (APCI)⁺ at m/z 392 (M+H)⁺, Rₜ=4.05 min.

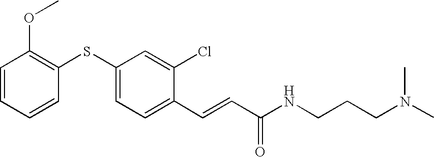

### EXAMPLE 24

### (E)-3-(2-chloro-4-(2-methoxyphenythiophenyl)-N-(3-(dimethylamino)propyl)acrylamide

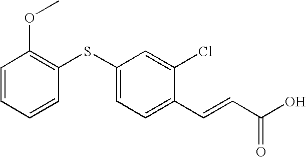

### EXAMPLE 24A

### (E)-3-(2-chloro-4-(2-methoxyphenylthio)phenyl)acrylic acid

Example 24 was prepared from the reaction of 2-methoxyobenzenethiol and 2-chloro-4-fluorobenzaldehyde followed by condensation with malonic acid according to the procedure described by Marty Winn et al., J. Med. Chem. 2001, 44(25), 4393-4403.

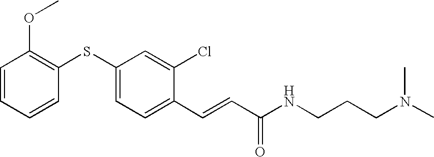

### EXAMPLE 24B

### (E)-3-(2-chloro-4-(2-methoxyphenylthio)phenyl)-N-(3-(dimethylamino)propyl)acrylamide

Example 24B was prepared from Example 24A as described for Example 1B, except substituting furan-3-yl(piperazin-1-yl)methanone with N^{I},N^{I}-dimethylpropane-1,3-diamine. LCMS (APCI)⁺ at m/z 405 (M+H)⁺, Rₜ=2.456 min.

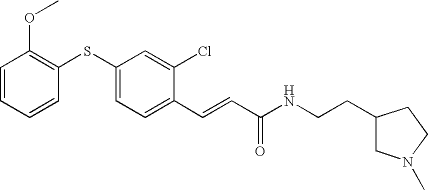

### EXAMPLE 25

### (E)-3-(2-chloro-4-(2-methoxyphenylthio)phenyl)-N-(2-(1-methylpyrrolidin-3-yl)ethyl)acrylamide

Title compound was prepared from Example 24A as described for the preparation of example 1B except substituting furan-3-yl(piperazin-1-yl)methanone with 2-(1-methylpyrrolidin-3-yl)ethanamine. LCMS (APCI)⁺ at m/z 431 (M+H)⁺, Rₜ=2.515 min.

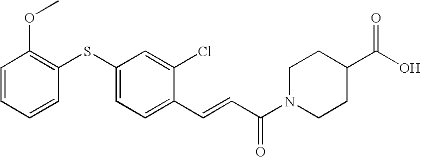

### EXAMPLE 26

### (E)-1-(3-(2-chloro-4-(2-methoxyphenylthio)phenyl)acryloyl)piperidine-4-carboxylic acid

Example 26949 mg) was prepared from Example 24A as described for Example 6. LCMS (APCI)⁻ at m/z 430 (M−H)⁻, Rₜ=2.84 min.

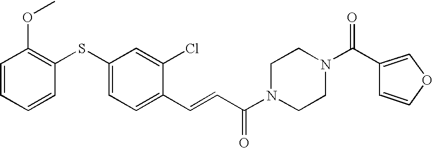

### EXAMPLE 27

### (E)-3-(2-chloro-4-(2-methoxyphenylthio)phenyl)-1-(4-(furan-3-carbonyl)piperazin-1-yl)prop-2-en-1-one

Example 24A was processed as in Example 1B to provide the title compound. LCMS (APCI)⁻ at m/z 483 (M−H)⁻, Rₜ=3.65 min.

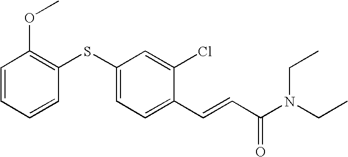

### EXAMPLE 28

### (E)-3-(2-chloro-4-(2-methoxyphenylthio)phenyl)-N,N-diethylacrylamide

Example 28 was prepared according to the method described for Example 18. LCMS (APCI)⁻ at m/z 376 (M−H)⁻, Rₜ=4.08 min.

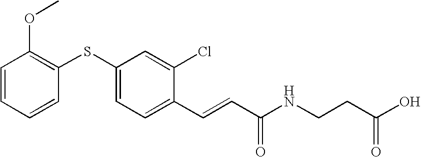

### EXAMPLE 29

### (E)-3-(3-(2-chloro-4-(2-methoxyphenylthio)phenyl)acrylamido)propanoic acid

Example 29 was prepared from Example 24A according to the procedure described for Example 20. LCMS (APCI)⁻ at m/z 390 (M−H)⁻, Rₜ=2.54 min.

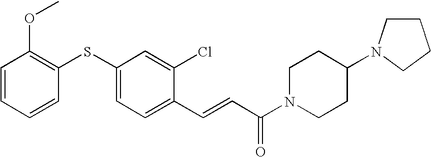

### EXAMPLE 30

### (E)-3-(2-chloro-4-(2-methoxyphenylthio)phenyl)-1-(4-(pyrrolidin-1-yl)piperidin-1-yl)prop-2-en-1-one

Example 30 was prepared from Example 24A as described for Example 1B except substituting furan-3-yl(piperazin-1-yl)methanone with 4-(pyrrolidin-1-yl)piperidine. LCMS (APCI)⁺ at m/z 457, 459 (M+H)⁺, Rₜ=2.94 min.

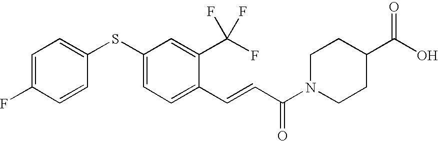

### EXAMPLE 31

### (E)-1-(3-(4-(4-fluorophenylthio-2-(trifluoromethyl)phenyl)acryloyl)piperidine-4-carboxylic acid

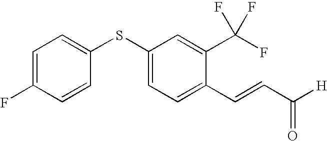

### EXAMPLE 31A

### (E)-3-(4-(4-fluorophenylthio)-2-(trifluoromethyl)phenyl)acrylaldehyde

Example 31A was prepared from the reaction of 4-fluorobenzenethiol with 4-fluoro-2-(trifluoromethyl)benzaldehyde followed by condensation with malonic acid according to the procedure described for Example 1A.

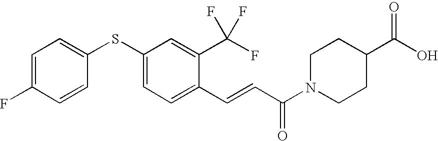

### EXAMPLE 31B

### (E)-1-(3-(4-(4-fluorophenylthio)-2-(trifluoromethyl)phenyl)acryloyl)piperidine-4-carboxylic acid

Title compound (47 mg) was prepared from Example 31A according to the procedure described for Example 6. LCMS (APCI)⁻ at m/z 452 (M−H)⁻, Rₜ=2.88 min.

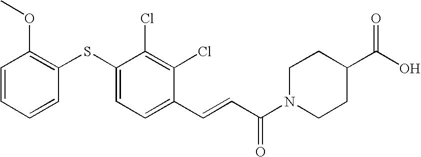

### EXAMPLE 32

### (E)-1-(3-(2,3-dichloro-4-(2-methoxyphenylthio)phenyl)acryloyl)piperidine-4-carboxylic acid

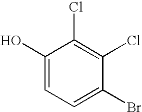

### EXAMPLE 32A

### 4-bromo-2,3-dichlorophenol

2,3-Dichlorophenol was treated with Br₂ in CH₂Cl₂ according to the method described in WO/00139081. LCMS (APCI−) at m/e 241 (M+H)⁺.

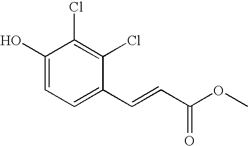

### EXAMPLE 32B

### (E)-methyl 3-(2,3-dichloro-4-hydroxyphenyl)acrylate

Example 32A was treated with methylacrylate in the presence of Pd₂(dba)₃, (Tol)₃P, triethylamine and in dry, DMF (300 mL) as described in WO/00139081. ¹H NMR (400 MHz, DMSO-d₆) δ 11.27 (s, 1H), 7.83 (d, J=16.01 Hz, 1H), 7.77 (d, J=8.98 Hz, 1H), 6.97 (d, J=8.98 Hz, 1H), 6.53 (d, J=16.01 Hz, 1H), 3.69 (s, 3H).

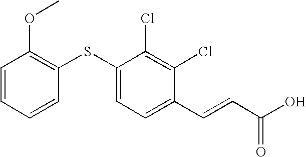

### EXAMPLE 32C

### (E)-3-(2,3-dichloro-4-(2-methoxyphenylthio)phenyl)acrylic acid

Example 32B was treated with 2,2,2-trifluoroacetic anhydride according to a literature protocol (WO/00139081) to obtain the corresponding triflate. Then the product isolated was treated with 2-methoxybenzenethiol as described in WO/00139081. Next the product isolated was processed as in Example 16F to provide the title compound. ¹H NMR (400 MHz, DMSO-d₆). δ 12.65 (br s, 1H), 7.83 (d, J=16.01 Hz, 1H), 7.74 (d, J=8.59 hz, 1H), 7.61-7.56 (m, 1H), 7.53-7.51 (m, 1H), 7.25 (d, J=8.20 Hz, 1H), 7.11-7.07 (m, 1H), 6.53-6.50 (m, 1H), 6.51 (d, J=167.01 Hz, 1H).

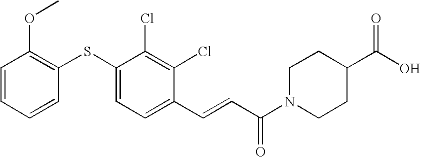

### EXAMPLE 32D

### (E)-1-(3-(2,3-dichloro-4-(2-methoxyphenylthio)phenyl)acryloyl)piperidine-4-carboxylic acid

Example 32C was treated with isonipecotic acid according to the procedure described for Example 5. Then the product isolated was treated with LiOH.H₂O as in Example 6 to provide the title compound. (400 MHz, DMSO-d₆) δ 12.26 (br s, 1H), 7.79 (d, J=8.59 Hz, 1H), 7.69 (d, J=16.01 Hz, 1H), 7.56-7.51 (m, 1H), 7.45-7.43 (m, 1H), 7.23-7.20 (m, 2H), 7.06-7.03 (m, 1H), 6.51 (d, J=8.98 Hz, 1H), 4.27-4.21 (m, 1H), 4.10-4.04 (m, 1H), 3.76 (m, 3H), 3.27 (m, 1H), 3.18-3.08 (m, 1H), 2.85-2.78 (m, 1H), 1.83-1.77 (m, 2H), 1.45-1.35 (m, 2H).

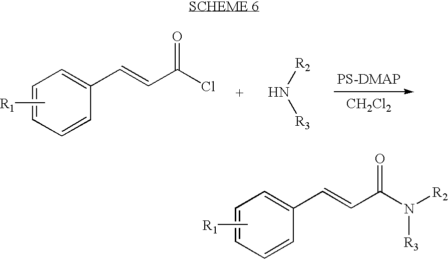

### General Procedure:

A solution of substituted phenylacryloyl chloride (2.50 mmol) in CH₂Cl₂ (2 ml) was added amine (2.75 mmol) followed by PS-DMAP (2.50 mmol) and stirred at ambient temperature for weekend. Reaction mixture filtered and concentrated to give title compounds in 70-90% yield in high purity.

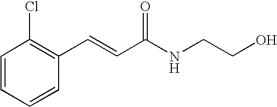

### EXAMPLE 33

### (E)-3-(2-chlorophenyl)-N-(2-hydroxyethyl acrylamide

LC-MS: m/z 226.959 (M+1)

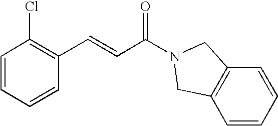

### EXAMPLE 34

### (E)-3-(2-chlorophenyl)-1-(isoindolin-2-yl)prop-2-en-1-one

LC-MS: m/z 285.197 (M+1)

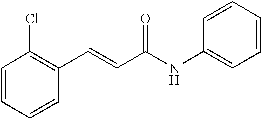

### EXAMPLE 35

### (E)-3-(2-chlorophenyl)-N-phenylacrylamide

LC-MS: m/z 259.133 (M+1)

TABLE 4

| | TR-FRET | TR-FRET |
|---|---|---|
| Example | IC₅₀* | IC₅₀* |
| Example 1B | A | A |
| Example 2 | B | B |
| Example 3 | A | A |
| Example 4 | A | A |
| Example 5 | A | A |
| Example 6 | A | A |
| Example 7 | B | B |
| Example 8 | B | B |
| Example 9B | B | B |
| Example 10 | A | A |
| Example 11 | A | A |
| Example 12 | A | A |
| Example 13 | A | A |
| Example 14 | A | A |
| Example 15 | A | A |
| Example 16G | A | A |
| Example 17 | A | A |
| Example 18 | A | A |
| Example 19 | A | A |
| Example 20 | A | A |
| Example 21 | B | B |
| Example 22 | A | B |
| Example 23 | A | A |
| Example 24B | A | A |
| Example 25 | A | A |
| Example 26 | A | A |
| Example 27 | A | A |
| Example 28 | A | A |
| Example 29 | A | A |
| Example 30 | A | A |
| Example 31B | A | A |
| Example 32D | A | A |
| Example 33 | A | A |
| Example 34 | A | A |
| Example 35 | A | A |

*A = 50-10 μM B < 10 μM

### HCV Helicase TR-FRET Unwinding Assay

Compound potency was assessed by determining the ability of the compound to inhibit DNA unwinding in an in vitro homogeneous time-resolved fluorescence quench assay. The helicase substrate (Perkin Elmer, TruPoint Helicase Substrate) consisted of partially double-stranded DNA, with one oligonucleotide strand labeled with a fluorescent europium chelate and the other strand labeled with the QSY™ 7 quencher. In the presence of helicase and ATP, this DNA is unwound and a large increase in fluorescence is observed. An excess of an unlabeled oligonucleotide (also from Perkin Elmer, TruPoint Helicase Capture Strand) that is complementary to the quencher strand was included in the assay to prevent reannealing of the europium and QSY-labeled strands.

The assay buffer consisted of 25 mM MOPS (pH 7.0), 500 μM MgCl₂, and 0.005% (v/v) Triton X-100, with DMSO being present at a final concentration of 2% (v/v). Recombinant, purified, full-length NS3 (1-631) protein was included in these assays at a final concentration of 2.5 nM. Compound was incubated with NS3 protein for 5 minutes in a 384-well white Proxiplate™ (Perkin Elmer) prior to the addition of TruPoint Helicase Substrate (4 nM final concentration), TruPoint Helicase Capture Strand (15 nM final concentration), and ATP (100 μM final concentration). The final reaction volume was 20 μL. Immediately after the addition of the substrates and capture strand, the initial rates of the unwinding reactions were determined at room temperature via an Envision (Perkin Elmer) plate reader. The rates of reactions containing test compound were compared to those lacking test compound in order to evaluate compound potency. IC₅₀ values were determined using the curve-fitting software XLfit (IDBS).

### NS3-NS4 Protease Assay

NS3 Complex Formation with NS4A-2

Recombinant E. coli or Baculovirus full-length NS3 was diluted to 3.33 μM with assay buffer and the material transferred to an eppendorf tube and place in water bath in 4° C. refrigerator. The appropriate amount of NS4A-2 to 8.3 mM in assay buffer was added to equal the volume of NS3 in step 2.1.1 (conversion factor−3.8 mg/272 μL assay buffer). The material was transferred to an eppendorf tube and placed in a water bath in a 4° C. refrigerator.

After equilibration to 4° C., equal volumes of NS3 and NS4A-2 solutions were combined in an eppendorf tube, mixed gently with a manual pipettor, and the mixture incubated for 15 minutes in the 4° C. water bath. Final concentrations in the mixture were 1.67 μM NS3, 4.15 mM NS4A-2 (2485-fold molar excess NS4A-2).

After 15 minutes at 4° C., the NS3/NS4A-2 eppendorf tube was removed and placed in a room temperature water bath for 10 minutes. NS3/NS4A-2 was aliquoted at appropriate volumes and store at −80° C. (E. coli NS3 run at 2 nM in assay, aliquot at 25 μL. BV NS3 Run at 3 nM in Assay, Aliquot at 30 μL).

### NS3 Inhibition Assay

Step 2.2.5. Sample compounds were dissolved to 10 mM in DMSO then diluted to 2.5 mM (1:4) in DMSO. Typically, compounds were added to an assay plate at 2.5 mM concentration, yielding upon dilution a starting concentration of 50 microM in the assay inhibition curve. Compounds were serial diluted in assay buffer to provide test solutions at lower concentrations.

Step 2.2.6. The E. coli NS3/NS4A-2 was diluted to 4 nM NS3 (1:417.5 of 1.67 μM stock−18 μL 1.67 μM stock+7497 μL assay buffer).

The BV NS3/NS4A-2 was diluted to 6 nM NS3 (1:278.3 of 1.67 μM stock −24 μL 1.67 μM stock+6655 μL assay buffer).

Step 2.2.7. Using the manual multichannel pipettor, and being careful not to introduce bubbles into the plate, 50 μL assay buffer was added to wells A01-H01 of a black Costar 96-well polypropylene storage plate.

Step 2.2.8. Using the manual multichannel pipettor, and being careful not to introduce bubbles into the plate, 50 μL of diluted NS3/NS4A-2 from step 2.2.6 was added to wells A02-H12 of plate in step 2.2.7.

Step 2.2.9. Using the manual multichannel pipettor, and being careful not to introduce bubbles into the plate, 25 μL of the wells in drug dilution plate in step 2.2.5 were transferred to corresponding wells in assay plate in step 2.2.8. The tips on multichannel pipettor were changed for each row of compounds transferred.

Step 2.2.10. Using the manual multichannel pipettor, and being careful not to introduce bubbles into the plate, the wells from the assay plate in step 2.2.9 were mixed by aspirating and dispensing 35 μL of the 75 μL in each well five times. The tips on multichannel pipettor were changed for each row of wells mixed.

Step 2.2.11. The plate was covered with a polystyrene plate lid, and the plate from step 2.2.10 containing NS3 protease and sample compounds was pre-incubated 10 minutes at room temperature.

While the plate from step 2.2.11 was pre-incubating, the RETS1 substrate was diluted in a 15 mL polypropylene centrifuge tube. The RETS1 substrate was diluted to 8 μM (1:80.75 of 646 μM stock−65 μL 646 μM stock+5184 μL assay buffer).

After the plate in step 2.2.11 was done pre-incubating, and using the manual multichannel, 25 μL of substrate was added to all wells on the plate. The contents of the wells were quickly mixed, as in step 2.2.10, but mixing 65 μL of the 100 μL in the wells.

The plate was read in kinetic mode on the Molecular Devices SpectraMax Gemini XS plate reader. Reader settings: Read time: 30 minutes, Interval: 36 seconds,

Reads: 51, Excitation λ: 335 nm, Emission λ: 495 nm, cutoff: 475 nm, Automix: off, Calibrate: once, PMT: high, Reads/well: 6, Vmax pts: 21 or 28/51 depending on length of linearity of reaction.

IC₅₀s were determined using a four parameter curve fit equation, and converted to Ki's using the following Km's:

Full-length E. coli NS3−2.03 μM

Full-length BV NS3−1.74 μM

where Ki=IC₅₀/(1+[S]/Km))

### Quantitation by ELISA of the Selectable Marker Protein, Neomycin Phosphotransferase II (NPTII) in the HCV Sub-Genomic Replicon, GS4.3

The HCV sub-genomic replicon (1377/NS3-3′, accession No. AJ242652), stably maintained in HuH-7 hepatoma cells, was created by Lohmann et al. Science 285: 110-113 (1999). The replicon-containing cell culture, designated GS4.3, was obtained from Dr. Christoph Seeger of the Institute for Cancer Research, Fox Chase Cancer Center, Philadelphia, Pa.

GS4.3 cells were maintained at 37° C., 5% CO₂, in DMEM (Gibco 11965-092) supplemented with L-glutamine 200 mM (100×) (Gibco25030-081), non-essential amino acids (NEAA) (Biowhittaker 13-114E), heat-inactivated (HI) Fetal Bovine Serum (FBS) (Hyclone SH3007.03) and 750 μg/ml geneticin (G418) (Gibco 10131-035). Cells were sub-divided 1:3 or 4 every 2-3 days.

24 hours prior to the assay, GS4.3 cells were collected, counted, and plated in 96-well plates (Costar 3585) at 7500 cells/well in 100 μl standard maintenance medium (above) and incubated in the conditions above. To initiate the assay, culture medium was removed, cells were washed once with PBS (Gibco 10010-023) and 90 μl Assay Medium (DMEM, L-glutamine, NEAA, 10% HI FBS, no G418) was added. Inhibitors were made as a 10× stock in Assay Medium, (3-fold dilutions from 10 μM to 56 μM final concentration, final DMSO concentration 1%), 10 μl were added to duplicate wells, plates were rocked to mix, and incubated as above for 72 h.

An NPTII ELISA kit was obtained from AGDIA, Inc. (Compound direct ELISA test system for Neomycin Phosphotransferase II, PSP 73000/4800). Manufacturer's instructions were followed, with some modifications. 10×PEB-1 lysis buffer was made up to include 500 μM PMSF (Sigma P7626, 50 mM stock in isopropanol). After 72 h incubation, cells were washed once with PBS and 150 μl PEB-1 with PMSF was added per well. Plates were agitated vigorously for 15 minutes, room temperature, then frozen at −70° C. Plates were thawed, lysates were mixed thoroughly, and 100 μl were applied to an NPTII Elisa plate. A standard curve was made. Lysate from DMSO-treated control cells was pooled, serially diluted with PEB-1 with PMSF, and applied to duplicate wells of the ELISA plate, in a range of initial lysate amount of 150 ul-2.5 ul. In addition, 100 μl buffer alone was applied in duplicate as a blank. Plates were sealed and gently agitated at room temperature for 2 h. Following capture incubation, the plates were washed 5×300 μl with PBS-T (0.5% Tween-20, PBS-T was supplied in the ELISA kit). For detection, a 1× dilution of enzyme conjugate diluent MRS-2 (5×) was made in PBS-T, into which 1:100 dilutions of enzyme conjugates A and B were added, as per instructions. Plates were resealed, and incubated with agitation, covered, room temperature, for 2 h. The washing was then repeated and 100 μl of room temperature TMB substrate was added. After approximately 30 minutes incubation (room temperature, agitation, covered), the reaction was stopped with 50 μl 3M sulfuric acid. Plates were read at 450 nm on a Molecular Devices Versamax plate reader.

Inhibitor effect was expressed as a percentage of DMSO-treated control signal, and inhibition curves were calculated using a 4-parameter equation: y=A+((B−A)/(1+((C/x)̂D))), where C is half-maximal activity or EC₅₀.

wherein:

A indicates an IC50 or EC50, as indicated, of less than 50 μM

B indicates an IC50 or EC50, as indicated, of less than 10 μM

C indicates an IC50 or EC50, as indicated, of less than 1 μM

and D indicates an IC50 or EC50, as indicated, of less the 0.1 μM

### CONCLUSION

Potent small molecule inhibitors of the HCV NS3 helicase have been developed.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A compound of the formula (I): 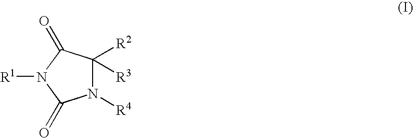
wherein:
R¹ is an optionally substituted aryl, an optionally substituted heterocyclyl comprising at least one of N, O or S, optionally substituted arylalkyl, or an optionally substituted heterocyclylalkyl comprising at least one of N, O or S in the heterocyclyl system;
R², R³ and R⁴ are each individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₆ to C₂₀ arylalkyl, optionally substituted C₃ to C₂₀ cycloalkylalkyl, optionally substituted C₅ to C₂₀ heteroarylalkyl, optionally substituted C₃ to C₂₀ heterocycylalkyl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; or
at least two of R², R³ and R⁴ join to form a ring wherein the ring is an unsubstituted or substituted 3 to 20 membered ring, wherein the members of the ring are selected from the group consisting of carbon, nitrogen, oxygen, and sulfur;
wherein formula (I) does not include the following structure:

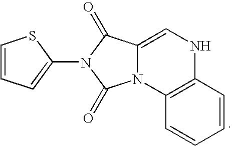

2. The compound of claim 1, wherein R¹ is an optionally substituted aryl or an optionally substituted heterocyclyl comprising at least one of N, O or S.

3. The compound of claim 1, wherein R², R³ and R⁴ are each individually selected from the group consisting of H, optionally substituted C₁ to C₂₀ alkyl, optionally substituted C₃ to C₂₀ partially saturated or fully saturated cycloalkyl, optionally substituted C₅ to C₂₀ aryl, optionally substituted C₆ to C₂₀ arylalkyl, optionally substituted C₃ to C₂₀ cycloalkylalkyl, optionally substituted C₅ to C₂₀ heteroarylalkyl, optionally substituted C₃ to C₂₀ heterocycylalkyl, optionally substituted C₁ to C₂₀ alkoxy, carbamyl, keto, carbonyl, carboxy, and combinations thereof.

4. The compound of claim 1, wherein at least two of R², R³ and R⁴ join to form a ring wherein the ring is an unsubstituted or substituted 3 to 7 membered ring, wherein the members of the ring are selected from the group consisting of carbon, nitrogen, oxygen, or sulfur.

5. The compound of claim 2, wherein R¹ is thiophene.

6. The compound of claim 5, wherein R², R³ and R⁴ are each individually selected from the group consisting of H, optionally substituted C₁ to C₂₀ alkyl, optionally substituted C₃ to C₂₀ partially saturated or fully saturated cycloalkyl, optionally substituted C₅ to C₂₀ aryl, optionally substituted C₆ to C₂₀ arylalkyl, optionally substituted C₃ to C₂₀ cycloalkylalkyl, optionally substituted C₅ to C₂₀ heteroarylalkyl, optionally substituted C₃ to C₂₀ heterocycylalkyl, optionally substituted C₁ to C₂₀ alkoxy, carbamyl, keto, carbonyl, carboxy, and combinations thereof.

7. The compound of claim 5, wherein at least two of R², R³ and R⁴ join to form a ring wherein the ring is an unsubstituted or substituted 3 to 7 membered ring, wherein the members of the ring are selected from the group consisting of carbon, nitrogen, oxygen, or sulfur.

8. The compound of claim 2, wherein R¹ is optionally substituted phenyl.

9. The compound of claim 8, wherein R², R³ and R⁴ are each individually selected from the group consisting of H, optionally substituted C₁ to C₂₀ alkyl, optionally substituted C₃ to C₂₀ partially saturated or fully saturated cycloalkyl, optionally substituted C₅ to C₂₀ aryl, optionally substituted C₆ to C₂₀ arylalkyl, optionally substituted C₃ to C₂₀ cycloalkylalkyl, optionally substituted C₅ to C₂₀ heteroarylalkyl, optionally substituted C₃ to C₂₀ heterocycylalkyl, optionally substituted C₁ to C₂₀ alkoxy, carbamyl, keto, carbonyl, carboxy, and combinations thereof.

10. The compound of claim 8, wherein at least two of R², R³ and R⁴ join to form a ring wherein the ring is an unsubstituted or substituted 3 to 7 membered ring, wherein the members of the ring are selected from the group consisting of carbon, nitrogen, oxygen, or sulfur.

11. The compound of claim 1 having a formula selected from I-1 to I-183.

12. A compound of the formula (II): 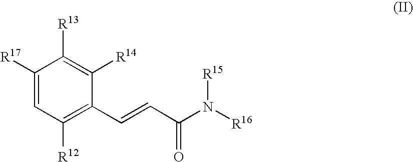
wherein:
R¹², R¹³, R¹⁴, and R¹⁷ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; wherein not all of R¹², R¹³, R¹⁴, and R¹⁷ are H;
R¹⁵ and R¹⁶ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₃ to C₂₀ heterocyclylalkyl, optionally substituted C₅ to C₂₀ heteroarylalkyl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, mono- and di-(C₁ to C₂₀)alkylamino, carbamyl, keto, carbonyl, carboxy, glycolyl, glycyl, hydrazino, guanylyl, and combinations thereof; or
R¹⁵ and R¹⁶ together form a ring wherein the ring is an unsubstituted or substituted 3 to 7 membered ring, wherein the members of the ring are selected from the group consisting of carbon, nitrogen, oxygen, or sulfur;
wherein formula (II) does not include the following structures:

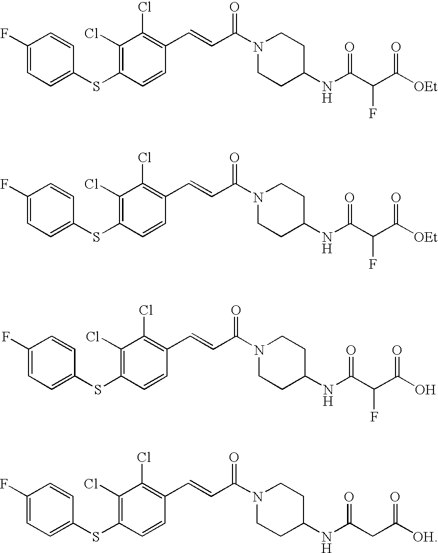

13. The compound of claim 12, wherein R¹², R¹³, R¹⁴, and R¹⁷ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₁ to C₂₀ alkoxy, optionally substituted C₁ to C₂₀ alkylthio, halo, cyano, mercapto, hydroxy, mono- and di-(C₁ to C₂₀)alkylamino, cyanoamino, nitro, carbamyl, keto, carbonyl, and carboxy.

14. The compound of claim 12, wherein R¹⁵ and R¹⁶ are individually selected from the group consisting of H, optionally substituted C₁ to C₂₀ alkyl, optionally substituted C₁ to C₂₀ alkenyl, optionally substituted C₁ to C₂₀ alkynyl, mono- and di-(C₁ to C₂₀)alkylamino, optionally substituted C₅ to C₂₀ aryl, optionally substituted C₃ to C₂₀ heterocyclylalkyl, optionally substituted C₅ to C₂₀ heteroarylalkyl, carbamyl, keto, carbonyl, carboxy, and combinations thereof.

15. The compound of claim 12, wherein R¹⁵ and R¹⁶ together form a ring wherein the ring is an unsubstituted or substituted 4 to 6 membered ring, wherein the members of the ring are selected from the group consisting of carbon, nitrogen, oxygen, and sulfur.

16. The compound of claim 12, having the formula (III): 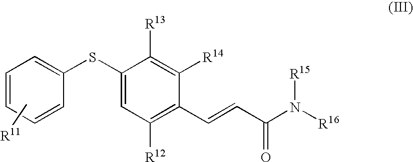
wherein:
R¹¹ is H, halo, optionally substituted C₁ to C₂₀ alkyl, optionally substituted C₁ to C₂₀ alkenyl, optionally substituted C₁ to C₂₀ alkynyl, or optionally substituted C₁ to C₂₀ alkoxy;
R¹², R¹³, and R¹⁴ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; wherein not all of R¹², R¹³, R¹⁴, and R¹⁷ are H;
R¹⁵ and R¹⁶ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₃ to C₂₀ heterocyclylalkyl, optionally substituted C₅ to C₂₀ heteroarylalkyl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, mono- and di-(C₁ to C₂₀)alkylamino, carbamyl, keto, carbonyl, carboxy, glycolyl, glycyl, hydrazino, guanylyl, and combinations thereof; or
R¹⁵ and R¹⁶ together form a ring wherein the ring is an unsubstituted or substituted 3 to 7 membered ring, wherein the members of the ring are selected from the group consisting of carbon, nitrogen, oxygen, or sulfur.

17. The compound of claim 16, wherein R¹¹ is H, halo, optionally substituted C₁ to C₂₀ alkyl, or optionally substituted C₁ to C₂₀ alkoxy.

18. The compound of claim 16, wherein R¹², R¹³, and R¹⁴ are individually selected from the group consisting of H, optionally substituted C₁ to C₂₀ alkyl, optionally substituted C₁ to C₂₀ alkoxy, optionally substituted C₁ to C₂₀ alkylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; wherein not all of R¹², R¹³, and R¹⁴ are H.

19. The compound of claim 16, wherein R¹², R¹³, and R¹⁴ are individually selected from the group consisting of H, optionally substituted C₁ to C₂₀ alkyl, optionally substituted C₁ to C₂₀ alkoxy, optionally substituted C₁ to C₂₀ alkylthio, halo, hydroxy, mono- and di-(C₁ to C₂₀)alkylamino, and combinations thereof; wherein not all of R¹², R¹³, and R¹⁴ are H.

20. The compound of claim 16, wherein R¹⁵ and R¹⁶ are individually selected from the group consisting of H, optionally substituted C₁ to C₂₀ alkyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₃ to C₂₀ heterocyclylalkyl, optionally substituted C₅ to C₂₀ heteroarylalkyl, optionally substituted C₁ to C₂₀ alkoxy, mono- and di-(C₁ to C₂₀)alkylamino, carbamyl, keto, carbonyl, carboxy, and combinations thereof.

21. The compound of claim 16, wherein R¹⁵ and R¹⁶ together form a ring wherein the ring is an unsubstituted or substituted 4 or 6 membered ring, wherein the members of the ring are selected from the group consisting of carbon, nitrogen, oxygen, or sulfur.

22. The compound of claim 16, wherein R¹¹ is fluoro and R¹², R¹³, and R¹⁴ are individually selected from the group consisting of H, alkyl, and halo.

23. The compound of claim 16 having a formula selected from II-1 to II-82.

24. A compound of claim 12 having the formula:

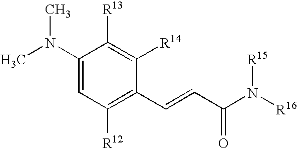

25. The compound of claim 24 having a formula selected from II-1 to II-82.

26. A compound of the formula (IV): 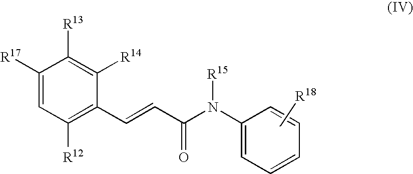
wherein:
R¹², R¹³, R¹⁴, and R¹⁷ are individually selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, halo, cyano, mercapto, hydroxy, mono- and di-(C₁ to C₂₀)alkylamino, cyanoamino, nitro, carbamyl, keto, carbonyl, carboxy, glycolyl, glycyl, hydrazino, guanylyl, sulfamyl, sulfonyl, sulfinyl, thiocarbonyl, thiocarboxy, and combinations thereof; wherein not all of R¹², R¹³, R¹⁴, and R¹⁷ are H;
R¹⁵ is selected from the group consisting of H, optionally substituted C₁ to C₂₀ alkyl, optionally substituted C₁ to C₂₀ alkenyl, optionally substituted C₁ to C₂₀ alkynyl, optionally substituted C₃ to C₂₀ partially saturated or fully saturated cycloalkyl, optionally substituted C₃ to C₂₀ partially saturated or fully saturated heterocyclic, optionally substituted C₅ to C₂₀ aryl, optionally substituted C₂ to C₂₀ heteroaryl, optionally substituted C₁ to C₂₀ heterocyclylalkyl, optionally substituted C₅ to C₂₀ heteroarylalkyl, optionally substituted C₁ to C₂₀ alkoxy, optionally substituted C₅ to C₂₀ aryloxy, optionally substituted C₁ to C₂₀ alkylthio, optionally substituted C₁ to C₂₀ arylthio, mono- and di-(C₁ to C₂₀)alkylamino, carbamyl, keto, carbonyl, carboxy, glycolyl, glycyl, hydrazino, guanylyl, and combinations thereof;
R¹⁸ is selected from the group consisting of H, optionally substituted C₁ to C₂₀ alkyl, optionally substituted C₁ to C₂₀ alkoxy, mono- and di-(C₁ to C₂₀)alkylamino, carbamyl, keto, carbonyl, carboxy, glycolyl, glycyl, hydrazino, guanylyl, and combinations thereof.

27. A pharmaceutical composition comprising a pharmaceutically acceptable excipient and a compound of claim 1.

28. A method of modulating NS3 activity comprising contacting an NS3 protein with an effective amount of a compound of claim 1.

29. The method of claim 28, wherein the contacting occurs ex vivo.

30. The method of claim 28, wherein the contacting occurs in vivo.

31. The method of claim 30, wherein the contacting occurs in a human body.

32. The method of claim 31, further comprising identifying a person having hepatitis C.

33. The method of claim 28, wherein the NS3 protein comprises a NS3 helicase domain.

34. The method of claim 28, comprising inhibiting NS3 helicase activity.

35. A compound comprising at least one functional group configured to facilitate binding of the compound to NS3 helicase, the binding being effective to modulate NS3 helicase activity.

36. The compound of claim 35, wherein the binding is effective to inhibit unwinding of a nucleic acid substrate by the NS3 helicase.

37. The compound of claim 36, wherein the nucleic acid substrate is DNA or RNA.

38. The compound of claim 35, wherein the binding facilitates allosteric movement of the NS3 helicase.

39. The compound of claim 35, wherein the functional group is configured to facilitate binding of the compound to NS3 helicase Domain 1.

40. The compound of claim 39, wherein the functional group is configured to facilitate binding of the compound to at least one residue in NS3 helicase Domain 1.

41. The compound of claim 40, wherein the residue is any one of Residues 209 to 221, Residues 286 to 288, Residues 317 to 319, or Residues 214 to 218.

42. The compound of claim 35, wherein the functional group is configured to facilitate binding of the compound to NS3 helicase Domain 2.

43. The compound of claim 42, wherein the functional group is configured to facilitate binding of the compound to at least one residue in NS3 helicase Domain 2.

44. The compound of claim 43, wherein the residue is any one of Residues 412 to 423, Residue 363, Residue 365, Residue 406, Residue 408, Residue 391, Residue 397, Residue 400, or Residues 400 to 404.

45. The compound of claim 35, wherein the modulating activity is inhibition.

46. The compound of claim 35, wherein the compound is any one of I-1 to I-183 and II-1 to II-82 as described in the specification.

47. A pharmaceutical composition comprising a compound of claim 35 and a pharmaceutically acceptable carrier.

48. The pharmaceutical composition of claim 47, wherein the compound is any one of I-1 to I-183 and II-1 to II-82 as described in the specification

49. A method of modulating NS3 helicase activity comprising contacting an NS3 protein with a compound of claim 35.

50. The method of claim 49, wherein the contacting occurs ex vivo.

51. The method of claim 49, wherein the contacting occurs in vivo.

52. The method of claim 51, wherein the contacting occurs in a human body.

53. The method of claim 52, further comprising a step of identifying a person having hepatitis C.

54-65. (canceled)

66. A pharmaceutical composition comprising a pharmaceutically acceptable excipient and a compound of claim 12.

67. A method of modulating NS3 activity comprising contacting an NS3 protein with an effective amount of a compound of claim 12.

68. The method of claim 67, wherein the contacting occurs ex vivo.

69. The method of claim 67, wherein the contacting occurs in vivo.

70. The method of claim 69, wherein the contacting occurs in a human body.

71. The method of claim 70, further comprising identifying a person having hepatitis C.

72. The method of claim 67, wherein the NS3 protein comprises a NS3 helicase domain.

73. The method of claim 67, comprising inhibiting NS3 helicase activity.

74. A pharmaceutical composition comprising a pharmaceutically acceptable excipient and a compound of claim 26.

75. A method of modulating NS3 activity comprising contacting an NS3 protein with an effective amount of a compound of claim 26.

76. The method of claim 75, wherein the contacting occurs ex vivo.

77. The method of claim 75, wherein the contacting occurs in vivo.

78. The method of claim 77, wherein the contacting occurs in a human body.

79. The method of claim 78, further comprising identifying a person having hepatitis C.

80. The method of claim 75, wherein the NS3 protein comprises a NS3 helicase domain.

81. The method of claim 75, comprising inhibiting NS3 helicase activity.
